(19) United States
(12) Patent Application Publication
(10) Pub. No.: US 2023/0221324 A1
(43) Pub. Date: Jul. 13, 2023

(54) BIOSENSING DEVICE FOR DETECTING CANCER

(71) Applicant: City University of Hong Kong, Hong Kong (CN)

(72) Inventors: Linlin LIU, Hong Kong (CN); Abhimanyu THAKUR, Hong Kong (CN); Wing Kar LI, Hong Kong (CN); Youngjin LEE, Hong Kong (CN); Chi-Man Lawrence WU, Hong Kong (CN)

(21) Appl. No.: 17/647,604

(22) Filed: Jan. 11, 2022

(51) Int. Cl. G01N 33/574; G01N 33/543

(52) U.S. Cl. CPC G01N 33/57488; G01N 33/54346; G01N 2333/70596; G01N 2333/705

(57) ABSTRACT

Disclosed herein are methods of detecting the presence or absence of exosomes, the method comprising detecting an exosomal biomarker in a sample obtained from a subject. Also disclosed herein is a system and a biosensor, each for detecting an exosomal biomarker as disclosed herein.

### FIELD OF THE INVENTION

The present invention relates generally to the field of molecular biology. In particular, the present invention relates to the use of biomarkers for the detection and diagnosis of cancer.

### BACKGROUND OF THE INVENTION

Glioblastoma (GBM) is an incurable brain tumour in which hypoxic GBM cells (GMs) increase the production and release of exosomes, 30-200 nm vesicles crossing the blood-brain-barrier, enabling the targeting of exosomal biomarkers for the tracking of GBM malignancy. However, conventional methods do not possess the sensitivity required in order to be able to detect exosomal biomarkers.

Due to lack of effective treatment options for tumours, such as, for example, glioblastomas, early detection of tumour formation and metabolic adaptation is necessary. Despite existing methods, including, for example, magnetic resonance imaging (MRI) as the gold standard for the diagnosis of cancers and tumours, for example glioblastomas (GBM), there is a demand of new techniques capable of detecting molecular and metabolic signatures of relevant organs, even at its early stage to aid in a more precise diagnosis.

Existing techniques used to diagnose glioblastoma rely on the use of methods such as clinical investigation, intracranial biopsies, and observation from imaging methods, including magnetic resonance imaging (MRI) and computed tomography (CT) scanning of the glioblastoma patient's brain. Despite extensive investigation, the detection of precise molecular signatures to monitor the development and malignant progression of glioblastoma has been difficult due to the limitations of available resources.

Thus, there is an unmet need for a method that is capable of detecting exosomal biomarkers.

### SUMMARY

In one aspect, the present disclosure refers to a method of detecting the presence or absence of an exosome in a sample, the method comprising contacting the sample suspected of comprising or comprising the exosome with a biosensor and detecting an exosomal biomarker on the exosome, wherein the biosensor is conjugated to a targeting agent selectively binding to and detecting the exosomal biomarker; wherein upon binding of the exosomal biomarker to the biosensor a signal is generated.

In another aspect, the present disclosure refers to a system for detecting an exosomal biomarker according to the method as disclosed herein, the system comprising a biosensor comprising nanoparticles localised on nano-islands, wherein the nanoparticles and the nano-islands are of the same or different noble metals, wherein the nanoparticles are conjugated to a targeting agent selectively binding to and detecting the exosomal biomarker, and a detector.

In yet another aspect, the present disclosure refers to a biosensor comprising metallic nanoparticles conjugated to a targeting agent, wherein the metallic nanoparticles are on the surface of metallic nano-islands, wherein the nanoparticles are conjugated to a targeting agent, and wherein the nanoparticles and the nano-islands are of the same or different noble metals.

### BRIEF DESCRIPTION OF THE DRAWINGS

The invention will be better understood with reference to the detailed description when considered in conjunction with the non-limiting examples and the accompanying drawings, in which:

FIG. 1 shows a schematic illustration of liquid biopsy of glioblastomas by the detection of enhanced MCT4 in blood-derived exosomes using Ag@AuNIs LSPR biosensor. FIG. 1A shows a schematic illustration of the Ag@AuNIs LSPR biosensor chip functionalized by biotinylated anti-MCT4 ABs for the detection of MCT4 in U87 GMs-derived exosomes. FIG. 1B and FIG. 1C show level of MCT4 and exosome release in hypoxic GMs in the TME are significantly upregulated for their growth and survival. The release of exosomes is enhanced, and exosomal MCT4 is also highly upregulated due to metabolic adaptation in GBM cells (GMs), supporting its importance in a functional mediator as well as a biomarker in GBM malignancy. Therefore, the label-free and sensitive detection of blood-derived exosomal MCT4 by an ultrasensitive biotinylated ABs functionalized (BAF) Ag@AuNIs LSPR biosensor introduces a novel biosensing method for tracking the malignancy of GBM as liquid biopsy.

FIG. 2 shows images of the characterization of the SAM Ag@AuNIs LSPR sensor chip. The surface morphology of the SAM Ag@AuNIs LSPR sensor chip examined by scanning electron microscopy (SEM) (FIG. 2A), high resolution transmission electron microscope (HR-TEM) (FIG. 2B) and atomic force microscopy (AFM) scanning (FIG. 2C-FIG. 2F). The surface of the sensor chip has circular or hexagonal Au nano-islands with the height and gap width of approximately 35-50 nm between adjacent nano-islands. To further investigate the nature between the localised Ag nanoparticles and Au nano-islands, a patch of Au nano-islands on the chip were deliberately scraped from the dielectric substrate and studied with HR-TEM. FIG. 2B demonstrated the side view of a Ag nano-particle locally attached on a Au nano-island, as indicated in a rectangular frame in FIG. 1A. It was observed that the Ag particle (light grey) was locally attached on the Au surface with clear boundary and contrast difference, confirming the Ag@AuNIs nanostructure. The interfacial distance of the Ag nanoparticle was found to be 0.235 nm with DigitalMicrograph software. This matches closely with the Ag(111) planes interfacial distance. The compositional profile of EDX line spectrum in FIG. 8 is consistent with Ag nanoparticles locally attached on Au nano-islands structure, in which Ag and Au atomic ratio was approximately 50% at the interface area, indicating that the interface is Au—Ag alloy. AFM phase scan was used to distinguish the nature of the Au nano-islands and the Ag nanoparticles, by scanning of the sensor chip surface nanostructure based on the hardness difference of the two materials. AFM scanning in height and phase mode at the same location (FIG. 2D-FIG. 2F) revealed the distinctive topographic image of the Au nano-island and the Ag nanoparticle of the nanostructure, as they have different stiffnesses. The surface of the Ag nanoparticles is selectively functionalized with biotinylated antibodies to detect antigen, while gaps between the Au nano-islands help to increase detection sensitivity (FIG. 2D-FIG. 2G). To test the performance of the SAM Ag@AuNIs LSPR sensor chip, the linear polarized light, including the s- and p-polarized light with sufficient retardation, was employed in the phase modulation system, to produce LSPR at the Ag@AuNIs-dielectric sensing interface in a total internal reflection scheme. In FIGS. 7A and 7B, a zero-mean spectral interferogram of the SAM Ag@AuNIs LSPR biosensor was given with the resonance point at 616.7 nm. The interference amplitude diminished dramatically at resonance wavelength because of the resonant transformation of photon into surface plasmon polariton on the SAM Ag@AuNIs LSPR sensor chip. To directly visualize the integrity and stability of nano-islands on the surface of the sensor chip, AFM tapping-mode was employed to produce topographic images to examine the surface of the sensor chip at various stages of biosensing. The surface of SAM Ag@AuNIs LSPR sensor chip was initially examined within a 1.5×1.5 μm² area, as shown in FIG. 2C. The nano-islands of the sensor chip were evenly distributed, and the root-mean-square surface roughness (Rq) was calculated as 8.04 nm (FIG. 9). After injection of biotinylated anti-MCT4 ABs for its functionalization, the sensor chip was flushed with PBS buffer and dried in nitrogen before AFM scanning of its surface. The flushing of the sensor chip with PBS buffer was to remove the non-specific bonded items and it is also to check the baseline of the binding affinity between sensor chip surface and target molecules. The Rq decreased to 6.46 nm (FIG. 9), indicating that there was a SAM biotinylated anti-MCT4 ABs layer formed at the gap of the nanostructure of sensor chip (FIG. 2G) and hence smoothened the surface. After incubation with exosomes, the sensor chip was taken off from the flow cell, further flushed with PBS buffer and dried in nitrogen to conduct AFM scanning There exist the unique distinctive spherical objects in the AFM topographic image of the surface of the sensor chip, further indicating that GMs-derived exosomes were immobilized on the SAM Ag@AuNIs LSPR sensor chip. The diameter of exosomes was found to be approximately 150 nm (FIG. 2H, FIG. 2I), and Rq was increased to 14.5 nm (FIG. 9), suggesting the successful capture of GMs-derived exosomes on the SAM Ag@AuNIs LSPR sensor chip, which was functionalized with biotinylated anti-MCT4 ABs (BAF Ag@AuNIs sensor chip).

FIG. 3 shows the results of characterization of glioblastoma (GMs)-derived exosomes and their detection by the biotinylated antibody functionalized self-assembly Ag nanoparticles locally attached on Au nano-islands (BAF Ag@AuNIs) localised surface plasmon resonance (LSPR) biosensor. Representative data of U87 glioblastoma (GMs)-derived exosomes is shown as follows in the following subfigures: FIG. 3A shows a graph of a representative calibration curve of the BAF Ag@AuNIs LSPR biosensor in the detection of the dynamic range of glioblastoma-derived exosome amount via CD63. FIG. 3B shows line graphs depicting the comparison of the detection sensitivity of glioblastoma (GMs)-derived exosomes by either BAF Ag@AuNIs- or functionalized gold-silver (Au—Ag) bimetallic-versus functionalized gold nano-island (AuNIs)-LSPR plasmonic biosensors. These graphs are based on linear regression equations for the best-fitting line of phase responses. FIG. 3C shows a graph depicting the size distribution detected by nanoparticle tracking analysis (NTA). FIG. 3D shows an image of the morphology and shape of the particles, obtained by transmission electron microscope (TEM). FIG. 3E shows images of CD63-positive dots stained by Immunogold electron microscopy (EM). FIG. 3F shows a representative image of MCT4-positive dots in glioblastoma (GMs)-derived exosomes stained by Immunogold electron microscopy (EM). FIG. 3G show a representative calibration curve of the BAF Ag@AuNIs LSPR biosensor for the detection of a dynamic range of glioblastoma (GMs)-derived exosome concentration via the MCT4 protein. FIG. 3H shows an image of Immunogold electron microscopy (EM) to check the MCT4 protein on retinal pigment epithelium (RPE) cell-derived exosomes. FIG. 3I) shows a graph depicting the results of comparative detection of MCT4 on glioblastoma (GMs)-derived exosomes and RPE cell-derived exosomes with anti-MCT4 antibody-functionalized SAM Ag@AuNIs LSPR biosensing. The data herein shows the characterisation of glioblastoma-derived exosomes in terms of its size, morphology, and shape by Nano-tracking Analysis (NTA) and transverse microscopy (TEM). Also, CD63 and MCT4 proteins were highly positive in glioblastoma-derived exosomes than RPE-derived exosomes, which served as a control. More importantly, from FIG. 3B, detection sensitivity of BAF Ag@AuNIs LSPR biosensor was higher than functionalized Au—Ag bimetallic nano-islands (BMNIs) and functionalized gold nano-islands (AuNIs). With a detection in glioblastoma-derived exosomes as shown in FIG. 3I, BAF Ag@AuNIs biosensors are shown to be able to detect glioblastoma-derived exosomes in vitro.

FIG. 4 shows data showing the detection of enhanced MCT4 in exosomes released from hypoxic glioblastoma cells (GMs) in the tumour microenvironment (TME). Subfigures FIG. 4A to FIG. 4H show representative images of the results of immunofluorescent staining for MCT4 in U87 glioblastoma cells (GMs) under normoxia and hypoxia (1% O₂). FIG. 4I shows a Western Blot result, indicating the change in protein level of MCT4 in U87 glioblastoma cells (GMs) in response to hypoxia (1% O₂). FIG. 4J shows a column graph showing the enhanced release of exosomes from hypoxic U87 glioblastoma cells (GMs) compared to normoxic U87 glioblastoma cells (GMs). FIG. 4K shows a graph providing representative phase responses of the LSPR biosensor with the functionalized SAM Ag@AuNIs sensing chip with anti-MCT4 antibodies in U87 glioblastoma (GMs)-derived exosomes under normoxic and hypoxic conditions. FIG. 4L shows the corresponding quantitative bar graph to FIG. 4K. The data herein shows that in the tumour microenvironment, tumour hypoxia is one of the hallmarks that were associated with poor prognosis in cancer patients, including malignant glioma patients. To better understand the capacity of BAF Ag@AuNIs LSPR biosensor in detecting levels of MCT4 from normoxic and hypoxic GMs-derived exosomes, U87 glioblastoma cells were exposed under normoxia and hypoxia (1% O₂) for comparisons. The level of MCT4 proteins was found to be augmented in hypoxic condition by both conventional methods (i.e., Western Blot and ELISA) and the method disclosed herein (BAF Ag@AuNIs biosensing by LSPR). To emphasize, with BAF Ag@AuNIs biosensing, only a small amount of sample (here, glioblastoma-derived exosomes) was required to give a sensitive detection of MCT4.

FIG. 5 shows the results of in vivo quantitative detection of MCT4 by BAF Ag@AuNIs LSPR biosensor to track progression of tumour. FIG. 5A shows images showing the results of in vivo bioluminescence imaging of mice injected with U87 glioblastoma cells (GMs), whereby the glioblastoma cells have been engineered to express luciferase. (B) shows a column graph depicting the quantitative total flux in sham-operated- and glioblastoma (GBM)-injected mouse at different concentrations with firefly luciferase. FIG. 5C shows a column graph showing tumour size-dependent exosome release.

FIG. 5D depicts a sensing calibration curve to show the dynamic range for the detection of MCT4 in glioblastoma (GBM) mouse blood exosomes by BAF Ag@AuNIs LSPR biosensor. FIG. 5E and FIG. 5H show representative LSPR phase response of 5 μg/ml and 0.5 μg/ml exosomes isolated from sham-operated and glioblastoma injected mice at different concentrations with firefly luciferase. FIG. 5F and FIG. 5I show correlation curve between level of U87-expressed luciferase total flux in sham-operated- and glioblastoma injected mice at different concentrations with firefly luciferase, and the strength of localised surface plasmon resonance (LSPR) responses to exosomal MCT4 [for 5 μg/ml, coefficient of determination (R²)=0.9736, and for 0.5 μg/ml, R²=0.9265]. FIG. 5G and FIG. 5J depict a correlation curve between concentration of exosomes released from sham-operated- and glioblastoma injected mice at different concentrations with firefly luciferase, and the strength of localised surface plasmon resonance (LSPR) responses to exosomal MCT4 [for 5 μg/ml, R²=0.8686, and for 0.5 μg/ml, R²=0.9341]. In this figure, it was determined whether increased exosomal MCT4 expression was also shown in in vivo U87 glioblastoma (GBM) mouse models compared to the sham-operated mice. By bioluminescence assay and NTA, increased size of glioblastoma, which was represented by total flux of luminescence in FIG. 5B, was found to be correlated with enhanced number of blood serum exosomes in glioblastoma mouse models. Coherently, in FIG. 5F, G, I, J, a positive correlation was also drawn between total flux and amount of exosome release, at concentrations of both 5 μg mL and 0.5 μg mL exosomes derived from blood serum of glioblastoma mouse models and sham-operated mice by BAF Ag@AuNIs biosensing. Moreover, a higher phase response of MCT4 was found in glioblastoma mouse models than sham-operated mice, which is positively correlated with increased total flux of luminescence and augmented exosome release as well.

FIG. 6 shows the results of monitoring changes in tumour size in glioblastoma (GBM) mice via the detection of MCT4 in blood serum exosomes using a BAF Ag@AuNIs LSPR biosensor. FIG. 6A show a schematic representation of the experimental design: 2 months old severe combined immunodeficiency (SCID) mice (n=3) were intracranially injected with luciferase-expressing U87 glioblastoma cells (GMs), followed by the bioluminescence imaging, isolation of blood serum exosomes, quantification of exosomal concentration, and quantification of MCT4 concentration in blood serum exosomes on day 8, 11, and 40 post-injection. FIG. 6B shows representative images of bioluminescence of luciferase-expressing U87 glioblastoma-injected mice. FIG. 6C shows column graphs showing the corresponding total flux. FIG. 6D shows column graph depicting the concentration of exosomes released from blood serum of the glioblastoma (GBM)-injected mice on day 8, 11, and 40 post-injection. FIG. 6E shows a graph depicting the results of the detection of MCT4 in exosomes released from blood serum of glioblastoma (GBM)-injected mice on day 8, 11, and 40 post-injection, while FIG. 6F shows the corresponding quantitative column graph. FIG. 6G shows a graph depicting the results of the detection of MCT4 in EGFRvIII immunocaptured exosomes released from blood serum of glioblastoma (GBM)-injected mice on day 8, 11, and 40 post-injection, while FIG. 6H shows the corresponding quantitative column graph. This data shows whether level of exosomal MCT4 expression in blood serum of GBM mouse models increased along with tumour growth in glioblastoma mouse models in a timescale manner, in order to test ability of the claimed BAF Ag@AuNIs biosensor in tracking tumour progression. It was found that localized surface plasmon resonance (LSPR) phase responses of MCT4 in glioblastoma mouse models increased along with total flux of luminescence and concentration of exosome release in a period of time, except a slight drop-in day 40 by the EGFRvIII-immunocaptured exosomes. This further affirms the practicality of BAF Ag@AuNIs biosensors in determining GBM malignancy and progression.

FIG. 7 shows experimental LSPR spectral interferograms with PBS solution being injected to the flow cell of SAM Ag@AuNIs LSPR biosensor in FIG. 7A between 550-750 nm, and in FIG. 7B zoom-in ranging between 590-640 nm of wavelength. The resonance point at 616.7 nm is evident and indicates better signal.

FIG. 8 shows images of the distances between the Au nanoisland and the Ag nanoparticle.

FIG. 8A is a HAADF-STEM image showing a part of a Au nano-island and a Ag nano-particle. The red line represents the EDX line scan from the Ag nanoparticle into Au nano-island so that in FIG. 8B gold content in red, and silver content in blue, in atomic ratio. The EDX scan along the line in FIG. 8A show that there was a Ag nano-particle locally attached on an Au nano-island. The interface area was Au—Ag alloy

FIG. 9 shows data of the determination of roughness to confirming the capture of GMs-derived exosomes on SAM Ag@AuNIs sensing chip. FIG. 9A shows a representative bar graph depicting the roughness of (bare SAM Ag@AuNIs sensing chip, FIG. 9B shows biotinylated anti-MCT4 AB functionalized to SAM Ag@AuNIs sensing chip, and FIG. 9C shows U87 GMs-derived exosomes captured on SAM Ag@AuNIs sensing chip via anti-MCT4 AB.

FIG. 10 graphs depicting the LSPR phase response depicting steps followed in the process of antibody function on different biosensing chips. FIG. 10A shows Au nano-islands (AuNIs) functionalization with 11-MUA and subsequently activated by EDC/HNS, then the anti-CD63 antibody was immobilized, FIG. 10B shows SAM Ag@AuNIs functionalized with biotinylated anti-MCT4 antibody and blocking with ethanolamine for the specific detection of exosomes. Similar method was followed in case of Au—Ag BMNIs sensing chip.

FIG. 11 shows images of the detection of MCT4 in blood serum-derived exosomes. FIG. 11A and FIG. 11B show the immunogold dots confirmed the presence of MCT4 in exosomes isolated from blood serum of sham-operated and GBM mouse model, respectively.

FIG. 12 shows a schematic of a biosensor as disclosed herein and its working within an exemplary system.

### DEFINITIONS

As used herein, the term “nano-island” refers to entities (droplets or other shapes) that are formed by, for example, spontaneous dewetting (also referred to as agglomeration) of thin and very thin metallic films on a substrate. Examples of dewetting include methods involving post-deposition heating or the use of other sources of energy.

As used herein, the term “nano-particles” refers to entities (droplets or other shapes) that ranges between 1 to 100 nanometres in size, that are formed by, for example, spontaneous dewetting (also referred to as agglomeration) of thin and very thin metallic films on a substrate. Examples of dewetting include methods involving post-deposition heating or the use of other sources of energy. The nanoparticles here is immobilized on the surface of nano-islands with size smaller than that of nano-islands.

As used herein, the term “hot spot” refers to a sharp nano roughness of plasmonic nanostructures. These hotspots usually occur at the edges or nanostructures.

As used herein, the term “localised surface plasmon resonance (LSPR)” refers to a specific sub-form of surface plasmon resonance. Surface plasmon resonance (SPR) refers to the resonant oscillation of conduction electrons at the interface between negative and positive permittivity material stimulated by incident light. LSPRs (localised surface plasmon resonances) refers to collective electron charge oscillations in metallic nanoparticles that are excited by light. In other words, in contrast to (propagating) surface plasmon resonance, localised surface plasmons (LSPs) are non-propagating excitations of the conduction electrons. These metallic nanoparticles exhibit enhanced near-field amplitude at the resonance wavelength. This field is highly localised at the nanoparticle and decays rapidly away from the nanoparticle/dielectric interface into the dielectric background, though far-field scattering by the particle is also enhanced by the resonance. Light intensity enhancement is a very important aspect of localised surface plasmon resonances and localisation means the localised surface plasmon resonances has very high spatial resolution (subwavelength), limited only by the size of nanoparticles. Because of the enhanced field amplitude, effects that depend on the amplitude such as magneto-optical effect are also enhanced by localised surface plasmon resonances. For nanosized silver and gold, the localised surface plasmon resonance falls into the visible range of the electromagnetic spectrum. Therefore, for example, silver or gold nanoparticles can be directly observed using, for instance, the dark-field scattering microscopy.

As used herein, the term “exosome” refers to extracellular vesicles that are released from cells upon fusion of an intermediate endocytic compartment, the multivesicular body (MVB), with the (cell) plasma membrane. This, in turn, liberates intraluminal vesicles (ILVs) into the extracellular milieu, resulting in the vesicles being released as exosomes.

As used herein, the term “normoxia” refers to external or ambient conditions at which the subject in question is under “normal” oxygen tension. This usually refers to ambient oxygen concentrations of 10% to 21%. Oxygen concentrations of below normoxia are usually referred to as being hypoxic oxygen tensions, or hypoxia, and usually refers to ambient oxygen concentrations of 1% to 5%. In the same line, hyperoxia refer to ambient oxygen concentrations of above 21%.

As used herein, the terms “Au” and “Ag” refer to the metals gold and silver, respectively.

As used herein, the term “targeting agent” refers to a molecule capable of binding to a target molecule. As appreciated by a person skilled in the art, such a targeting agent can be chosen in accordance with the intended target molecule. Examples of such targeting agents are, but are not limited to, antibodies and biotinylated antibodies. Examples of target molecules are, but are not limited to, one or more of the exosomal biomarkers disclosed herein.

### DETAILED DESCRIPTION OF THE PRESENT INVENTION

Glioblastoma (GBM) is a fatal and incurable brain tumour. Its late diagnosis at symptomatic stage using magnetic resonance imaging (MRI)- and computed tomography (CT)-scans, and final determination of tumour state by invasive biopsy is a significant hurdle to its therapy. Therefore, developing sensitive label-free and minimally- or non-invasive biosensor for the detection of early GBM formation and progression is in high demand for its effective diagnosis, prognosis, and therapy. During their malignant progression, GBM cells (GMs) in the hypoxic tumour microenvironment (TME) produces and releases detectable amounts of exosomes and lactate, partly through metabolic reprogramming, which is known as the Warburg effect. Accumulated evidence has revealed that GMs-derived exosomes containing pro-oncogenic components significantly promote tumour progression via playing cargo shuttles among neighbouring cells, and they also cross the blood-brain-barrier (BBB) into the circulating blood and systemically influence on long-distant cells, enabling them to be diagnostic biomarker-discovery platforms to track GBM progression as liquid biopsy.

Monocarboxylate transporter 4 (MCT4), a crucial lactate transporter in cancer, is dramatically upregulated in malignant hypoxic GMs with a hypoxia-inducible factor dependent manner to remove intracellular lactate, leading to the support of the survival and growth of cancer cells. Therefore, MCT4 has been known as a malignancy biomarker in cancer. Importantly, upregulated MCT4 has been identified in malignant GMs-derived exosomes which are also involved in the progression of GBM, implying that exosomal MCT4 can be also a biomarker to track the malignant progression of GBM. Due to lack of effective treatment options for tumours, such as, for example, glioblastomas, early detection of tumour formation and metabolic adaptation is necessary. Despite existing methods, including, for example, magnetic resonance imaging (MRI) as the gold standard for the diagnosis of cancers and tumours, for example glioblastomas (GBM), there is a demand of new techniques capable of detecting molecular and metabolic signatures of relevant organs, even at its early stage to aid in a more precise diagnosis. In one such examples, there is disclosed a method of detecting such metabolic or molecular changes in the glioma.

Existing techniques used to diagnose glioblastoma rely on the use of methods such as clinical investigation, intracranial biopsies, and observation from imaging methods, including magnetic resonance imaging (MRI) and computed tomography (CT) scanning of the glioblastoma patient's brain. Despite extensive investigations, the detection of precise molecular signatures to monitor the development and malignant progression of glioblastoma has been difficult due to the limitations of available resources. Thus, in one example, the biosensor as disclosed herein has been developed comprising silver (Ag) nanoparticles locally attached on gold (Au) nano-islands utilising localised surface plasmon resonance as a detection signal. As used herein, the term “localised” refers to the placement of nanoparticles on the surface of the nano-islands disclosed herein. The term “localised” may also be used interchangeably with “attached to” or “attached on”.

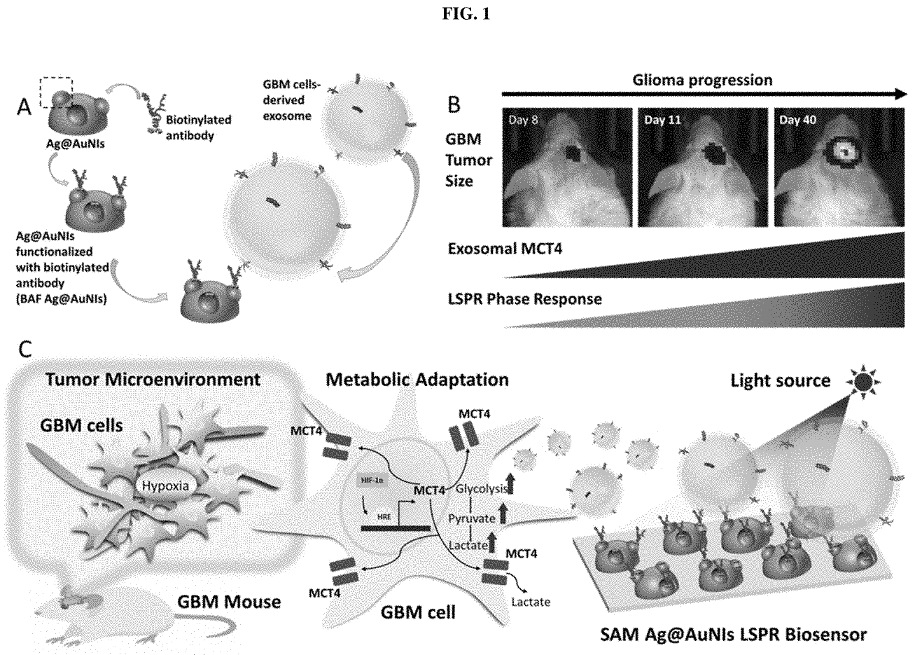

There are several available biosensing methods to characterize and quantify exosomes and exosomal proteins based on surface-enhanced Raman scattering (SERS), fluorescence, colorimetry and immunochromatographic assay (ICA). However, they have limitations to be a practical biosensing technique due to the requirement of expensive equipment or long process labelling. They also suffer from negative aspects of limited application to detect a surface protein, low sensitivity to detect a tiny amount of exosomal proteins, or difficulty to conduct the translation process in clinics. Therefore, a localized surface plasmon resonance (LSPR) biosensor with self-assembly silver nanoparticles locally attached on gold nano-islands (SAM Ag@AuNIs) sensor chip (SAM Ag@AuNIs LSPR biosensor) was established for the sensitive label-free detection of exosomal biomarkers, such as but not limited to, MCT4, for early diagnosis of glioblastoma formation and progression through metabolic adaptation (FIG. 1A-C).

A self-assembly, Ag nanoparticles locally attached on Au nano-islands (SAM Ag@AuNIs) nanostructure sensor chip were developed. The sensor has high detection sensitivity and selectivity with rational dynamic range and good stability in sensing with the synergistic effect of a unique Au—Ag nanostructure. Biotinylated antibodies were selectively immobilized on the Ag nanoparticles located on the surface of Au nano-islands due to strong adsorption of biotin with Ag nanoparticles (FIG. 1A, C). This functionalized sensor surface can capture analyte of exosomal biomarkers, thus enabling detection. With the Au nano-islands structure, the electromagnetic near-field strength is enhanced at the gaps of nano-islands, i.e., the “hot spots”, thus enhancing sensing performance. Here, a site-specific functionalization of biotinylated antibodies onto the Ag nanoparticles took advantage of enhanced electromagnetic (EM) near-field, as well as the plasmonic properties of Ag and Au, which resulted in enhanced sensitivity.

Furthermore, utilizing the SAM Ag@AuNIs LSPR sensor chip, the enhanced level of exosomal MCT4, a major glioblastoma malignancy biomarker, was detected in the blood of a mouse model of glioblastoma, providing an application of the chip disclosed herein in a diagnostic and/or prognostic method to determine, for example, the metabolic signature and malignant status of parent glioblastoma, as well as the effect of, for example, an anti-cancer agent in glioblastoma progression, using samples such as, but not limited to, liquid biopsies.

The present disclosure outlines the development and application of a localised surface plasmon resonance (LSPR) biosensor with a sensing chip comprising, for example, self-assembly silver (Ag) nanoparticles locally attached on gold (Au) nano-islands (SAM Ag@AuNIs), also referred to herein as “SAM Ag@AuNIs LSPR biosensor” (or chips), as well as biotinylated antibody functionalized (BAF) Ag@AuNIs LSPR biosensors or chips, also referred to herein as “BAF Ag@AuNIs LSPR biosensors.

Exemplary, non-limiting applications of the described biosensor are also disclosed herein. In one example, said biosensor is used for the detection of exosomes in a sample. In one example, such an exosome can be detected by way of biomarkers, for example, but not limited to exosome-specific or exosomal biomarkers.

### Characterization of the SAM Ag@AuNIs LSPR Sensor Chip

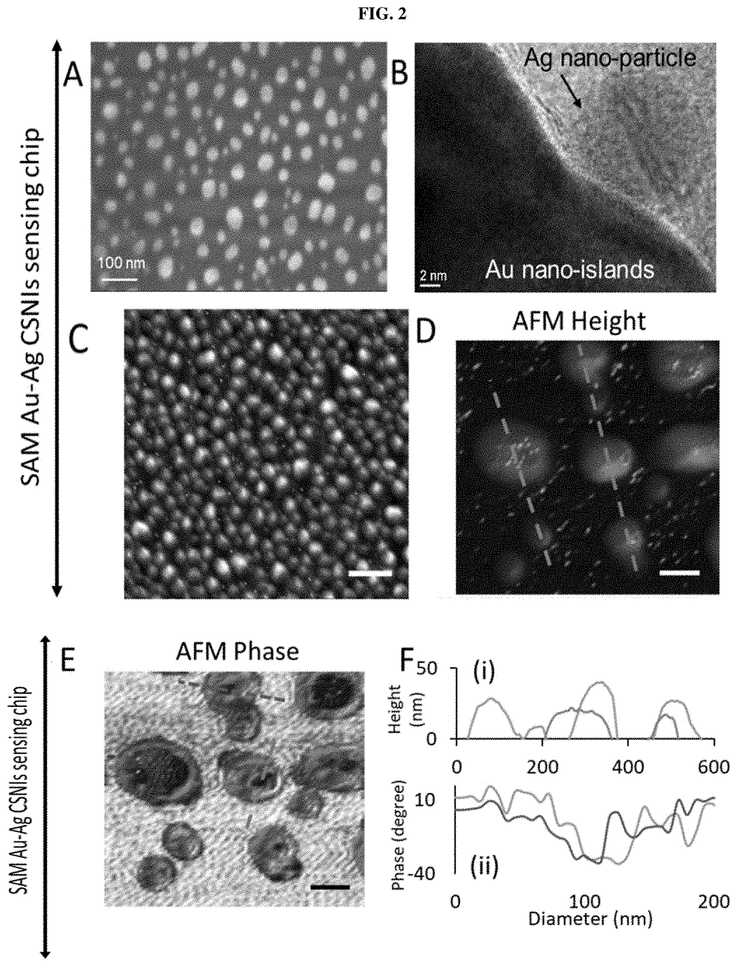

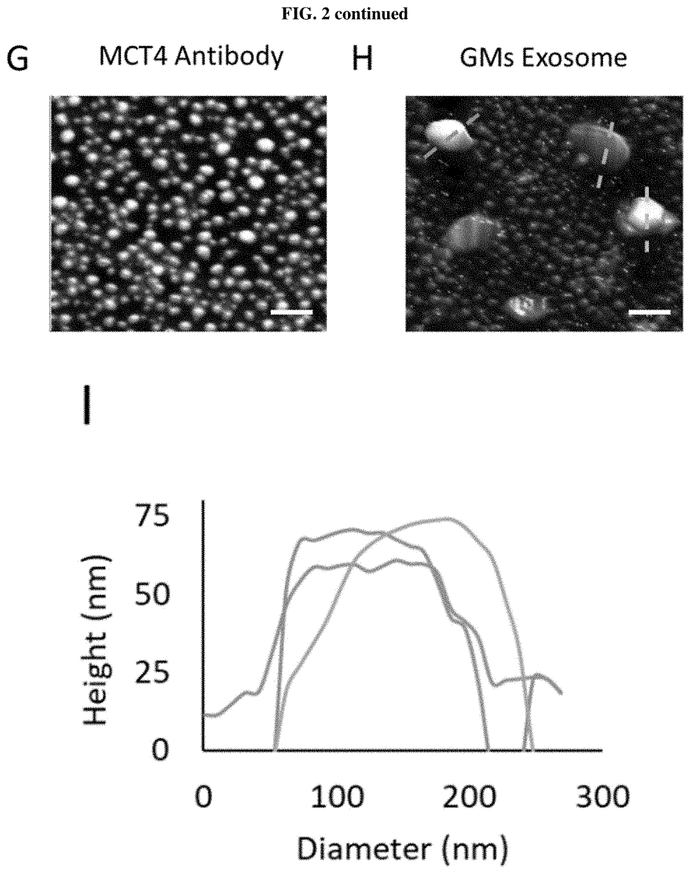

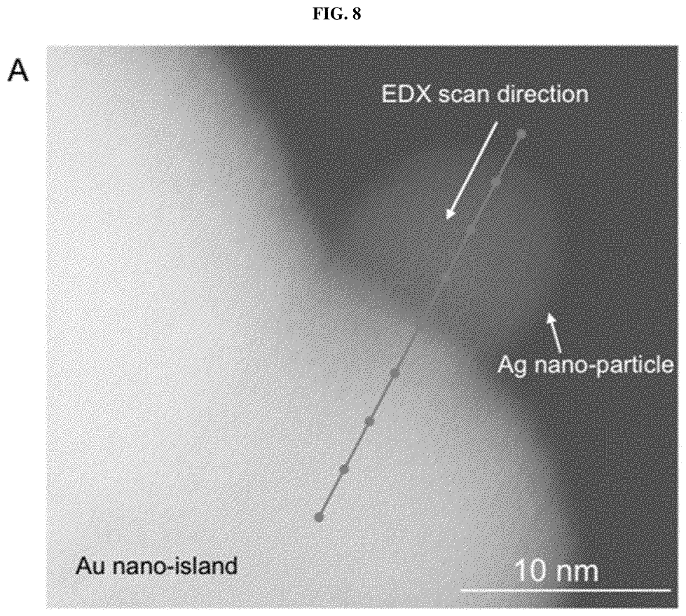

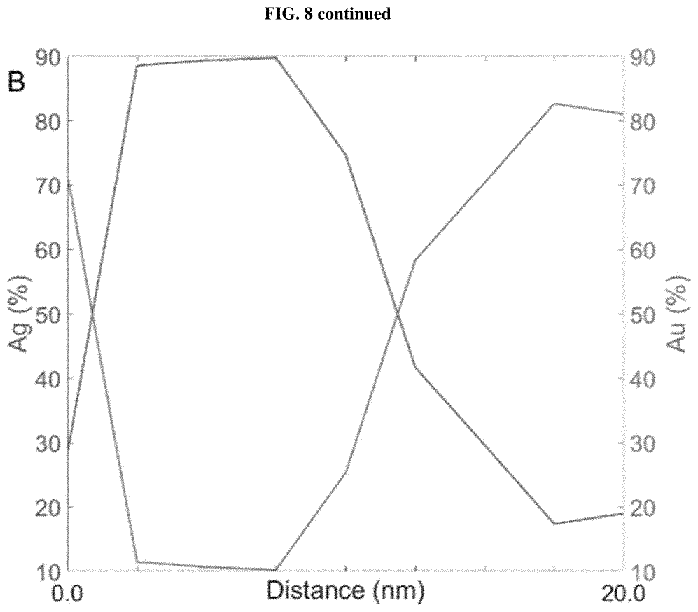

The surface morphology of the SAM Ag@AuNIs LSPR sensor chip was examined by scanning electron microscopy (SEM; FIG. 2A), high resolution transmission electron microscope (HR-TEM; FIG. 2B) and atomic force microscopy (AFM) scanning (FIG. 2C-F). The surface of the sensor chip has circular or hexagonal Au nanoislands. To further investigate the nature between the Ag nanoparticles locally attached on Au nanoislands, a patch of Au nano-islands on the chip was deliberately scraped from the dielectric substrate and studied with HR-TEM. FIG. 2B demonstrated the side view of an Ag nanoparticle locally attached on an Au nanoisland, as indicated in a rectangular frame in FIG. 1A. It was observed that the Ag nanoparticle was locally attached on the Au nano-island surface with clear boundary and contrast difference, confirming the Ag@AuNIs nanostructure. The interfacial distance of Ag nanoparticle was found to be 0.235 nm with the Digital Micrograph software. This matches closely with the Ag(111) planes interfacial distance. The compositional profile of EDX line spectrum in FIG. 8 is consistent with Ag nanoparticles locally attached on Au nano-islands structure, in which the Ag and Au atomic ratios were approximately 50% at the interface area, indicating that the interface is an Au—Ag alloy. AFM phase scan was used to distinguish the nature of the Au nano-islands and the Ag nanoparticles, by scanning of the sensor chip surface nanostructure based on the hardness difference of the two materials. AFM scanning in height and phase mode at the same location (FIG. 2D-F) revealed the distinctive topographic image of the Au nano-islands and the Ag nanoparticles of the nanostructure as they have different stiffnesses. The surface of the Ag nanoparticles is selectively functionalized with biotinylated ABs to detect antigen, while gaps between the Au nanoislands help to increase detection sensitivity.

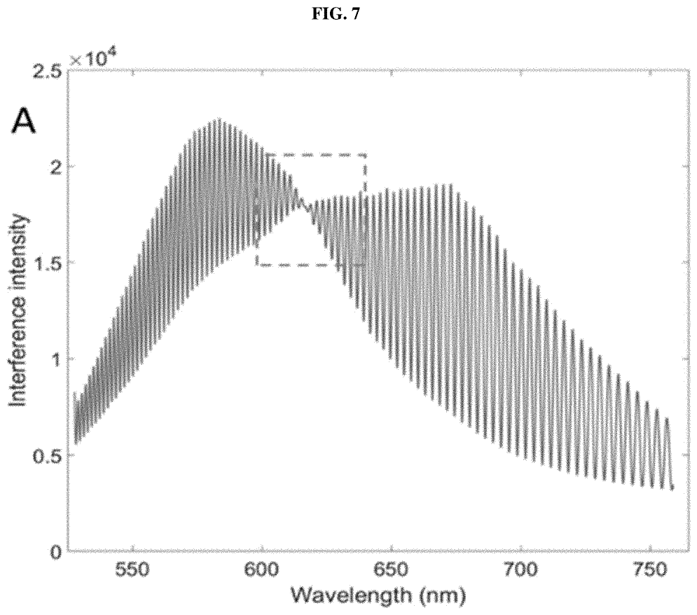

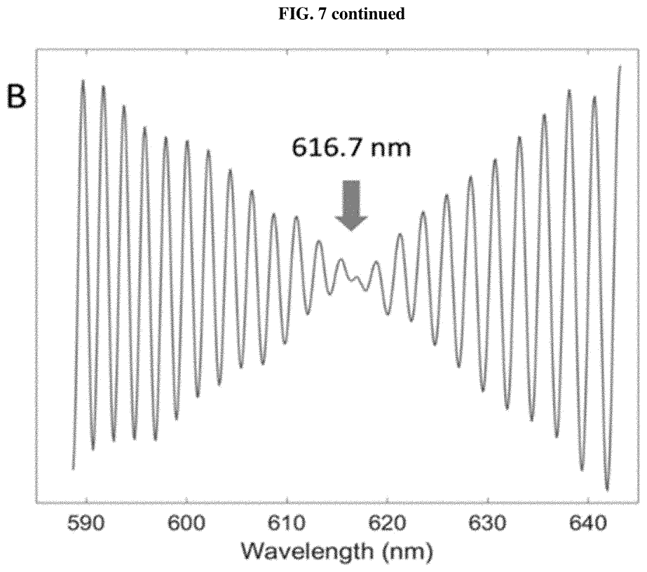

To test the performance of the SAM Ag@AuNIs LSPR sensor chip, the linear polarized light, including the s- and p-polarized light with sufficient retardation, was employed in the phase modulation system, to produce LSPR at the Ag@AuNIs-dielectric sensing interface in a total internal reflection scheme. In FIGS. 7A and 7B, a zero-mean spectral interferogram of the SAM Ag@AuNIs LSPR biosensor was given with the resonance point at 616.7 nm. The interference amplitude diminished dramatically at resonance wavelength because of the resonant transformation of photon into surface plasmon polariton on the SAM Ag@AuNIs LSPR sensor chip.

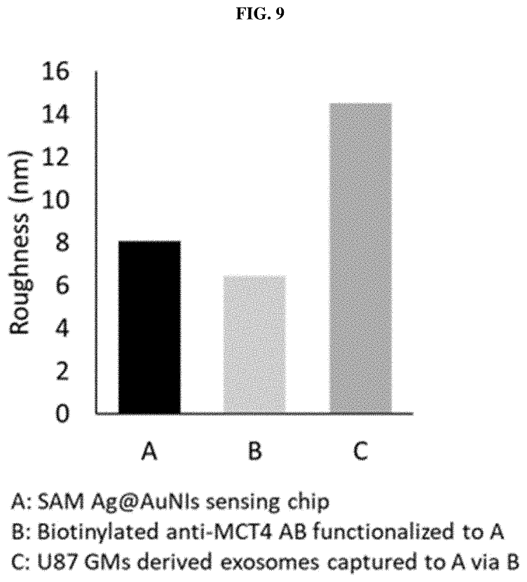

To directly visualize the integrity and stability of nanoislands on the surface of the sensor chip, AFM tapping-mode was employed to produce topographic images to examine the surface of the sensor chip at various stages of biosensing. The surface of SAM Ag@AuNIs LSPR sensor chip was initially examined within a 1.5×1.5 μm² area, as shown in FIG. 2C. The nanoislands of the sensor chip was evenly distributed, and the root-mean-square surface roughness (Rq) was calculated as 8.04 nm (FIG. 9). After injection of biotinylated anti-MCT4 ABs for its functionalization, the sensor chip was flushed with PBS buffer and dried in nitrogen before AFM scanning of its surface. The flushing of the sensor chip with PBS buffer was to remove the non-specific bonded items and it is also to check the baseline of the binding affinity between sensor chip surface and target molecules. The Rq decreased to 6.46 nm (FIG. 9), indicating that there was a SAM biotinylated anti-MCT4 ABs layer formed at the gap of the nanostructure of sensor chip (FIG. 2G) and hence smoothened the surface. After incubation with exosomes, the sensor chip was taken off from the flow cell, further flushed with PBS buffer and dried in nitrogen to conduct AFM scanning There exist the unique distinctive spherical objects in the AFM topographic image of the surface of the sensor chip, further indicating that GMs-derived exosomes were immobilized on the BAF Ag@AuNIs LSPR sensor chip. The diameter of exosomes was found to be approximately 150 nm (FIGS. 2H, I), and Rq was increased to 14.5 nm (FIG. 9), suggesting the successful capture of GMs-derived exosomes on the SAM Ag@AuNIs LSPR sensor chip which was functionalized with biotinylated anti-MCT4 ABs (BAF Ag@AuNIs sensor chip).

The biosensor described herein can be used as imaging agents in biological systems by, for example, but not limited to, covalent conjugation to a targeting agent that selectively binds to a specific organ, tissue, cell, cellular receptor, polynucleotide, lipid, polypeptide, carbohydrate, small molecule, etc. In certain embodiments, the compounds described herein are covalently conjugated to a targeting agent. The targeting agent can be an antibody, an antibody fragment (such as Fab, Fab′, F(ab′)2, and Fv), single chain (ScFv)) a peptide, an aptamer, or a small molecule that is capable of selectively binding to a target of interest, such as a carbohydrate, polynucleotide, lipid, polypeptide, protein, small molecule, cellular receptor, etc. Covalent conjugation of the biosensor described herein, and the targeting agent can be accomplished using well known methods known by the skilled person.

### Determination of the Sensitivity of the SAM Ag@AuNIs LSPR Sensor Chip to Detect a Tumour Biomarker, MCT4, in GMs-Derived Exosomes

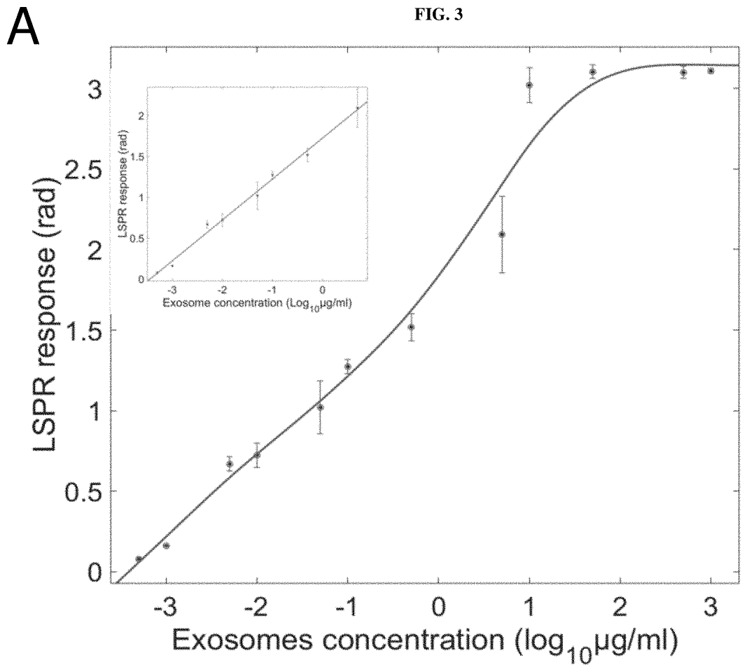

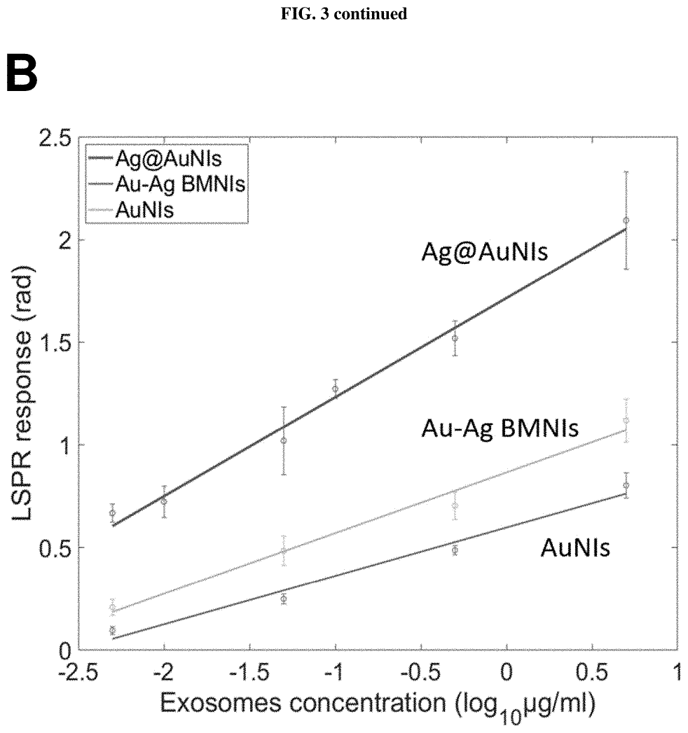

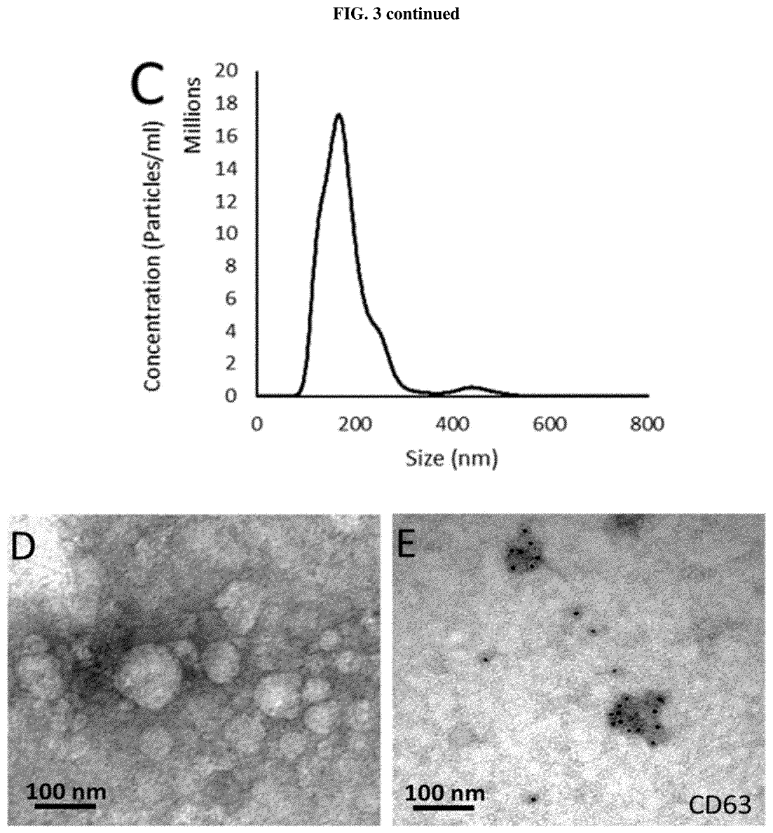

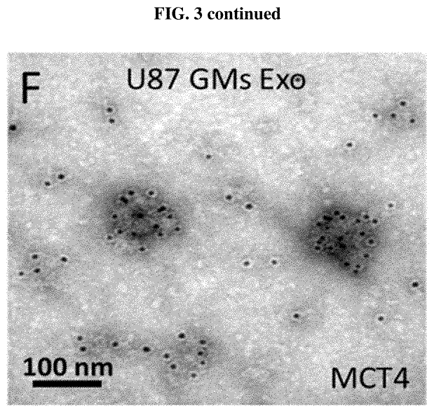

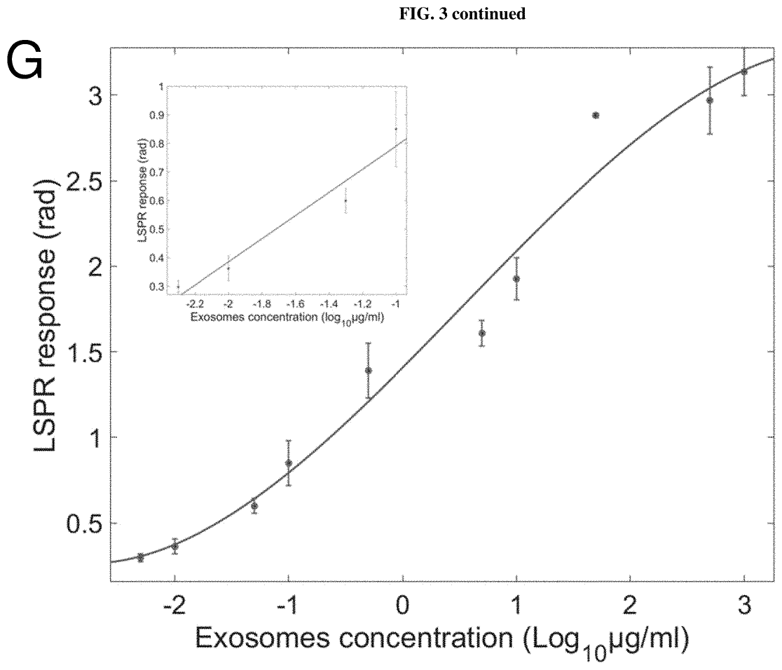

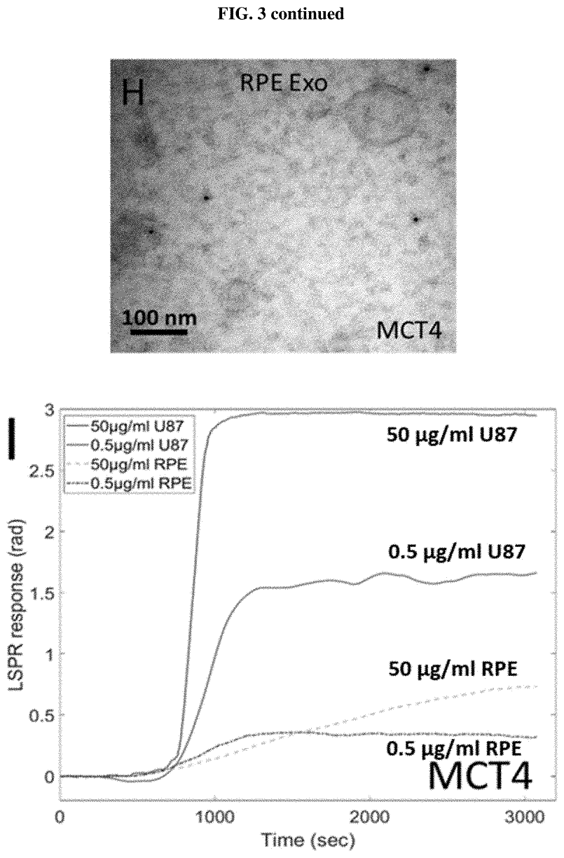

To investigate the sensing performance of the SAM Ag@AuNIs LSPR sensor chip in the detection of exosomal biomarkers, biotinylated ABs for CD63, an identification marker of exosomes, were first functionalized on the SAM Ag@AuNIs LSPR sensor chip. Next, various concentrations of GMs-derived exosomes were used to determine the dynamic range and the sensitivity of the detection of exosomal CD63. In detail, GMs-derived exosomes with concentrations in the range of 0.0005 to 1000 μg/mL were injected into the probe cell respectively, followed by PBS flushing in each test. LSPR phase responses were increased with an exosome-concentration dependent manner, as shown in FIG. 3A. When exosome concentration reached 50 μg/mL, the phase response was saturated at approximately 3.14 rad. Thus, the BAF Ag@AuNIs LSPR sensor chip exhibited a wide dynamic range of detection for exosomal CD63 from 0.0005 to 50 μg/mL exosome concentration, i.e., five orders of magnitude. The sensing calibration for exosomal CD63 in the range from 0.0005 μg/mL to 5 μg/mL exosome concentration was calculated, and the resultant formula of linear regression equation was established to be y=0.5003×x+1.724 (R² 0.9933). According to the definition by International Union of Pure and Applied Chemistry (IUPAC), limit of detection (LOD) of sensing system, the sum of the mean of blank measures, i.e., 0.0035 rad with PBS buffer and triple of its standard deviation, i.e., 3×(3.3×10³) rad, was determined. Thus, the uncertainty of current optical sensing system is (0.0035)+3×(3.3×10³)=0.0135 rad. Then, this uncertainty was substituted by the formula of linear calibration equation to calculate the minimum detectable concentration, which is known as the LOD. The LOD of BAF Ag@AuNIs LSPR sensor chip was found to be 3.8×10 μg/mL (FIG. 3A, B).

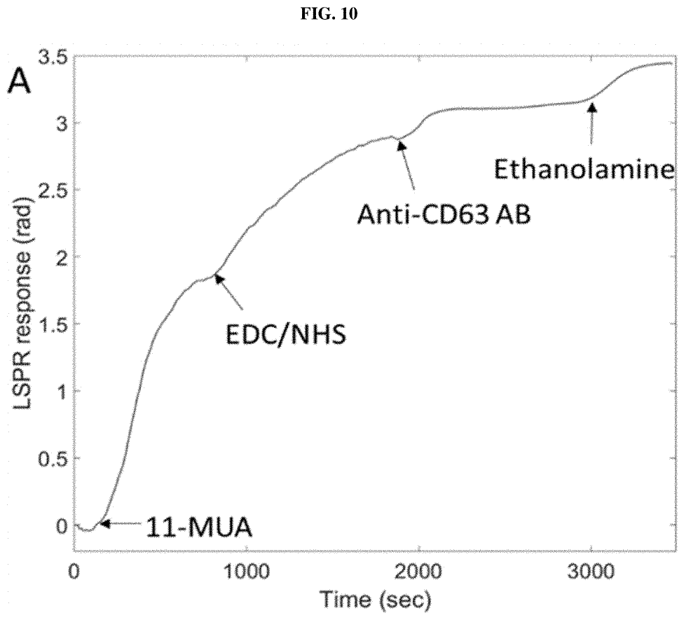

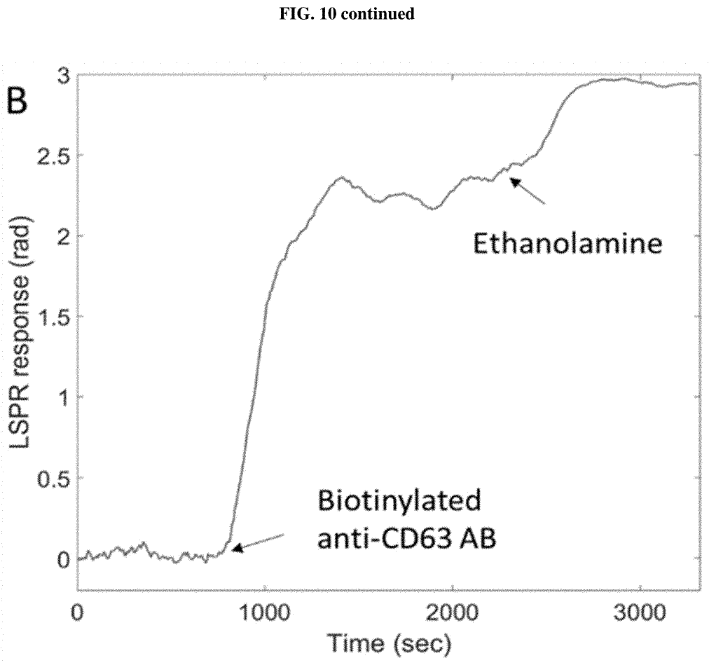

To compare the detection sensitivity of the proposed BAF Ag@AuNIs LSPR sensor chip with other Au nanoislands (AuNIs)-, and Au—Ag alloy bimetallic nanoislands (Au—Ag BMNIs)-chips, the phase response produced by each chip towards exosomal CD63 in the range of exosome concentration between 0.005 μg/mL and 5 μg/mL was obtained and compared. Each chip has a nanostructure with unique properties of metallic material. Thus, it has different functionalization processes with antibodies. For example, thiolates were found to be residues for the functionalization of Au based on the covalent sulphur-gold (RS—Au) interaction. Therefore, 11-mercaptoundecanoic acid (11-MUA) was used to functionalize the plasmonic AuNIs with ABs containing thiolates (FIG. 10A). However, the functionalization of Ag nanoparticles with biotinylated ABs was established owing to the direct interaction between Ag and biotin, reducing the steps involved in conventional functionalization processes. In terms of sensitivity, in the thiolate-functionalized AuNIs chip, the linear regression equation was found to be y=0.2925×x+0.8685 (R² 0.9818), and the LOD of the AuNIs chip was found to be 1.5×10⁻³ μg/mL exosome concentration. The linear regression equation for biotinylated ABs functionalized Au—Ag BMNIs sensor chip was found to be y=0.2358×x+0.598 (R²=0.9769), with the calculated LOD as 4×10⁻³ μg/mL exosome concentration. The sensitivity of the three sensor chips was compared by the linear regression curves as shown in FIG. 3B. The BAF Ag@AuNIs LSPR sensor chip exhibited much steeper slope than the other two, indicating that the BAF Ag@AuNIs LSPR sensor chip had the highest sensitivity, which is 1.71 and 2.12 times of those of the AuNIs and Au—Ag BMNIs chips, respectively.

Indeed, the spatial extent of the binding of biotinylated ABs with GMs-derived exosomes on the BAF Ag@AuNIs LSPR sensor chip was much closer than that of thiolate-Au functionalization scheme, in which the immobilized 11-MUA was necessary to be further activated by NHS/EDC (N-(3-dimethylaminopropyl)-N′-ethylcarbodiimide) before the immobilization of ABs (FIG. 10A, B). This could provide less reduction caused by binding length between the sensing surface receptors. Thus, the BAF Ag@AuNIs LSPR sensor chip was able to provide better LSPR sensitivity, as evidenced by the improvement of corresponding LOD (FIG. 3A, B).

The higher expression of MCT4 in tumour tissue is associated with the poor prognosis in GBM patients. Therefore, MCT4 level in glioblastoma is considered to be a biomarker for its diagnosis and prognosis. However, exosomal MCT4 has not been investigated as a glioblastoma biomarker. Exosomes isolated from U87 glioblastoma cells were found to be lying in the range of approximately 80-300 nm (FIG. 3C), and circular in shape (FIG. 3D). CD63 positive dots in immunogold EM demonstrated the presence of an exosomal marker (FIG. 3E), confirming exosome identity. Therefore, the SAM Ag@AuNIs LSPR biosensor was utilized to detect the presence and quantity of MCT4 in U87 GMs-derived exosomes together with immunogold EM (FIG. 3F). The dynamic range and limit of detection (LOD) of the biotinylated anti-MCT4 ABs functionalized Ag@AuNIs LSPR biosensor for MCT4 in U87 GMs-derived exosomes were 0.0014-500 μg/mL and 1.4 ng/mL, respectively (FIG. 3G) with the linear regression equation, y=0.4052×x+1.196 (R² 0.9468). These results suggested the capability of the BAF Ag@AuNIs LSPR biosensor to detect MCT4 in GMs-derived exosomes. In addition, the BAF Ag@AuNIs LSPR biosensor was proven for its good sensitivity for exosomal MCT4 in the phase responses to two different concentrations of exosomes at 0.5 and 50 μg/mL. The phase responses were found to increase at 800 seconds after the injection of exosomes in the sensing probe cell and they were stabilized at the amplitude of 2.956 rad and 1.649 rad, respectively, without reduction when PBS was flushed into the probe cell at 1200 seconds (FIG. 3F, H, I). As expected from the data of immunogold EM, retinal pigment epithelium (RPE) cell-derived exosomes which was known as MCT4-negative cells also produced weak phase responses (FIG. 3H), presumably by the non-specific interaction between anti-MCT4 ABs and exosomal MCT4. Interestingly, these nonspecific bindings were partially removed by PBS flushing and obvious dissociation occurred (FIG. 3I).

### The Sensitive Detection of MCT4 Enhancement in Malignant Hypoxic GMs-Derived Exosomes Using BAF Ag@AuNIs LSPR Biosensor

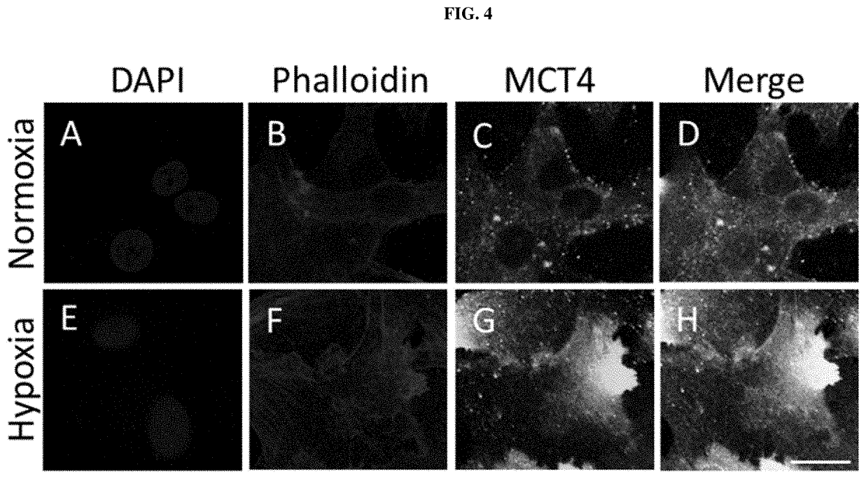

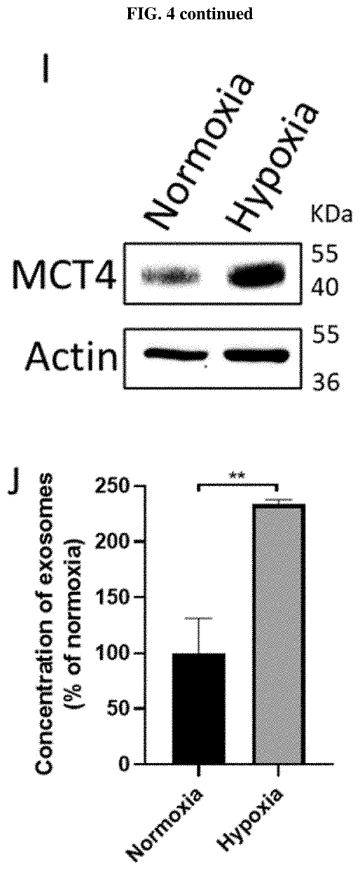

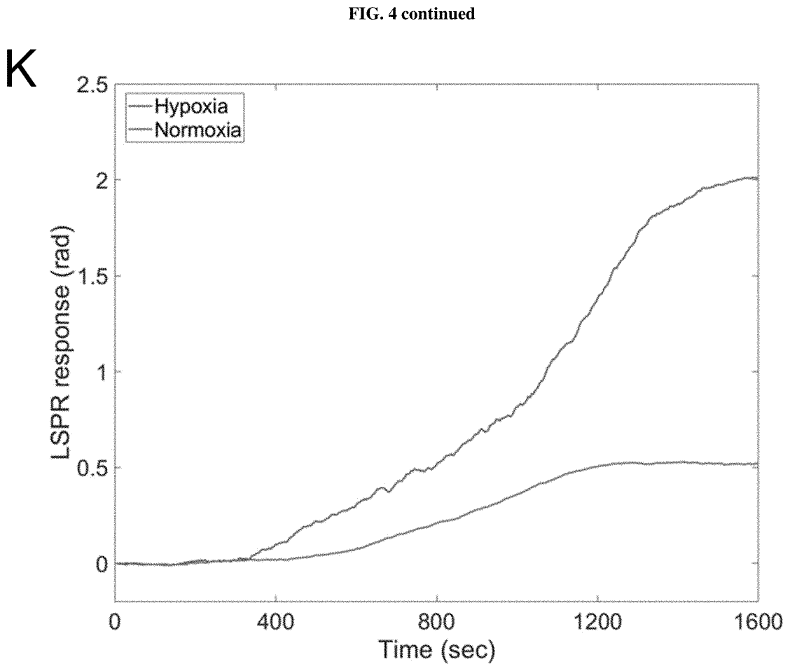

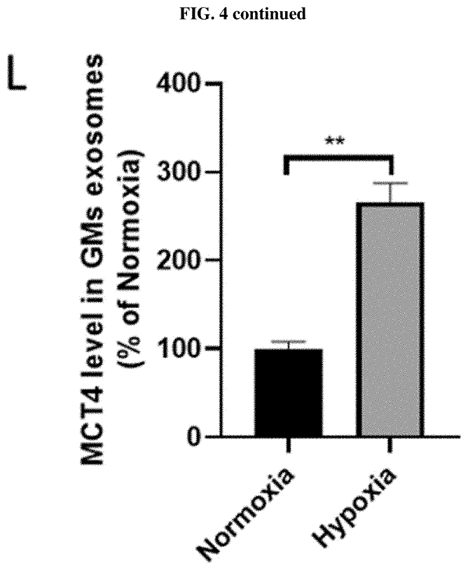

Hypoxia in tumour microenvironment (TME) is one of the major causatives to promote malignant tumour progression partly via upregulating glycolysis-related proteins, such as MCT4 in GMs. To examine the capability of the BAF Ag@AuNIs LSPR biosensor to determine the hypoxic and malignant state of GMs via detecting exosomal MCT4, U87 GMs were exposed to hypoxic-condition, and MCT4 levels in normoxic- and hypoxic GMs-derived exosomes were detected and compared. Under hypoxia, MCT4 was found to be enhanced on the membrane of U87 glioblastoma cells (FIG. 4A-H), and the total protein amount of MCT4 was also increased significantly in U87 glioblastoma cells under hypoxia compared to normoxia (FIG. 4I). Interestingly, exosome release was increased (around 25 times) from hypoxic glioblastomas compared to normoxic glioblastomas (FIG. 4J).

Conventional methods such as western blot and ELISA possess limitations for the detection of exosomal proteins because of requirement of high amount of sample, complicated processes and protein damaged during sample process, and less sensitivity. Therefore, the sensitive label-free BAF Ag@AuNIs LSPR biosensor was developed, which could detect MCT4 in small amounts of GMs-derived exosomes with limit of detection (LOD) of 1.4 ng/mL exosome concentration (FIG. 3F, G), and it could also detect specifically MCT4 in GMs-derived exosomes although slight non-specific phase response occurred in RPE-derived exosomes (FIG. 3H, I). Importantly, the LSPR phase response for MCT4 towards hypoxic GMs-derived exosomes (2.01 rad) was found to be significantly higher as compared to the normoxic GMs-derived exosomes (0.52 rad) (FIG. 4K, L), indicating that the BAF Ag@AuNIs LSPR biosensor can be used as the diagnostic tool for the detection of exosomal MCT4 to track the development as well as malignant progression of GBM.

### BAF Ag@AuNIs LSPR Biosensor Identified GBM Formation by Detecting Increased MCT4 Level in Blood Serum-Derived Exosomes of a Mouse Model.

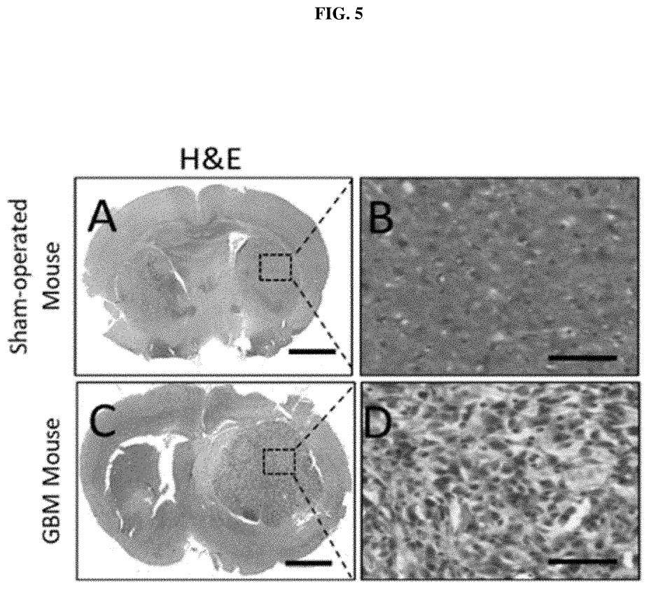

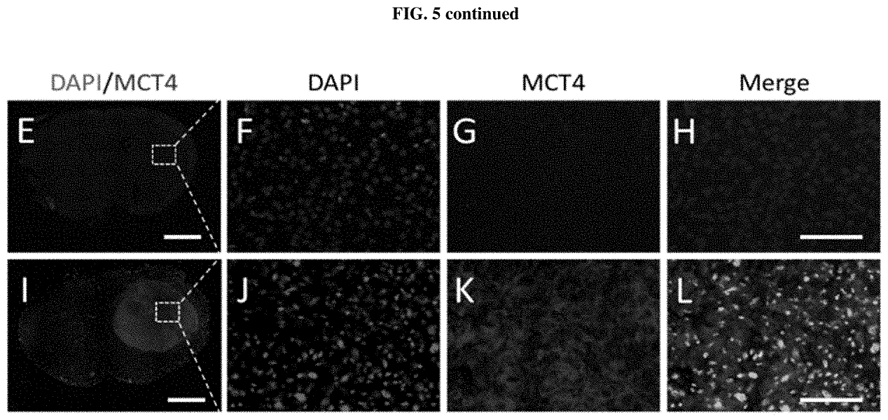

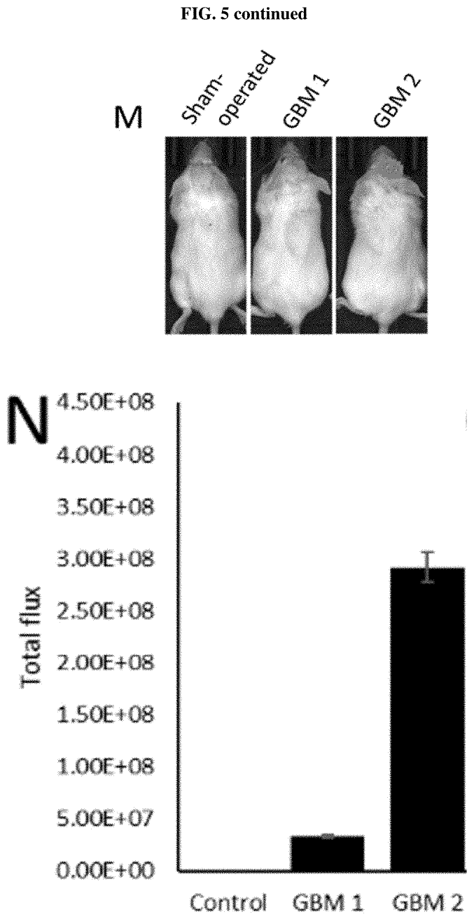

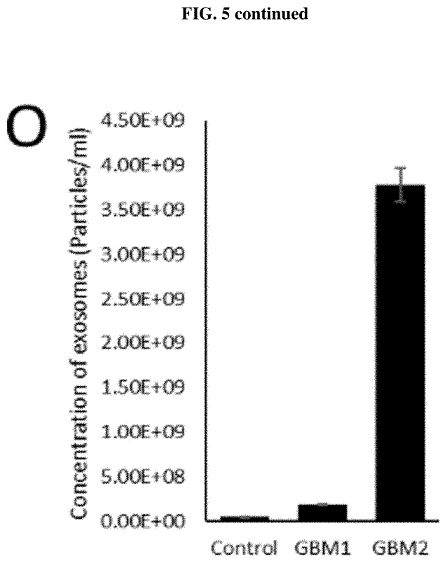

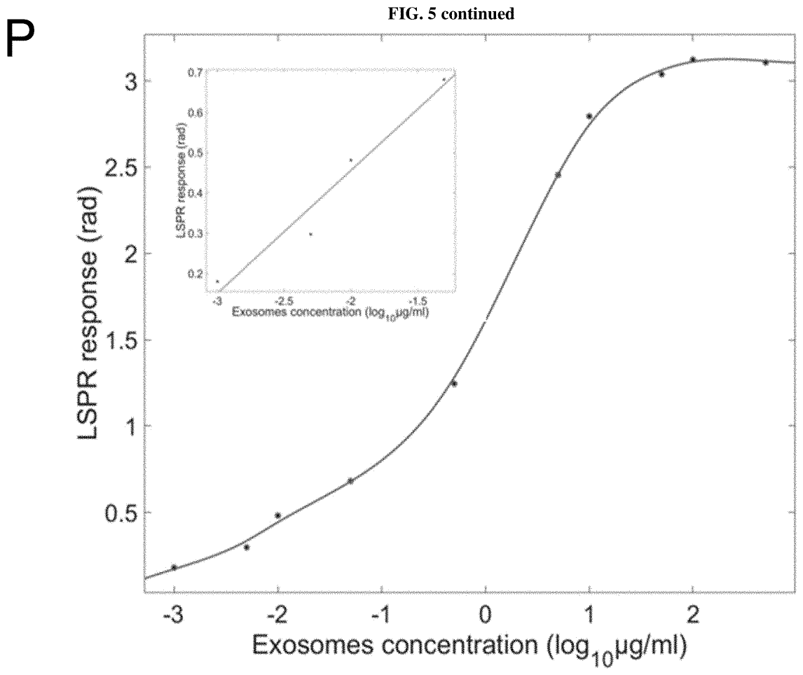

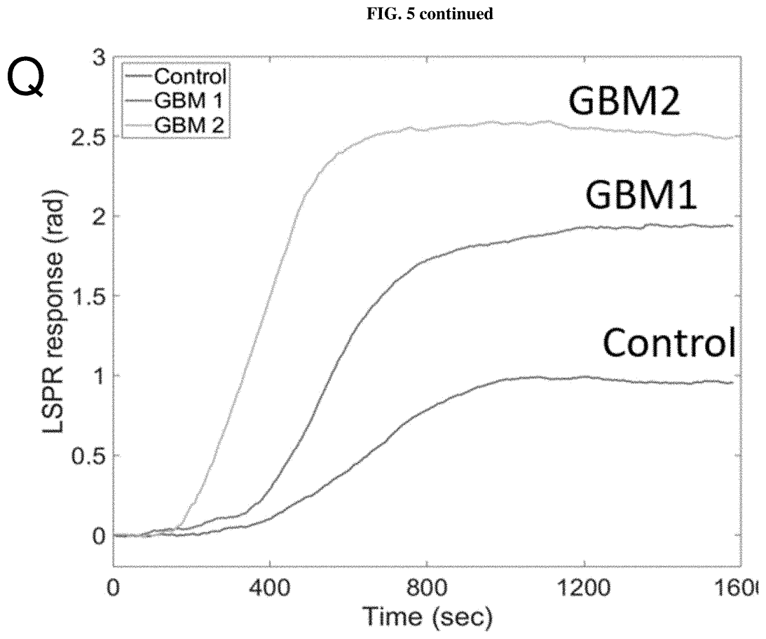

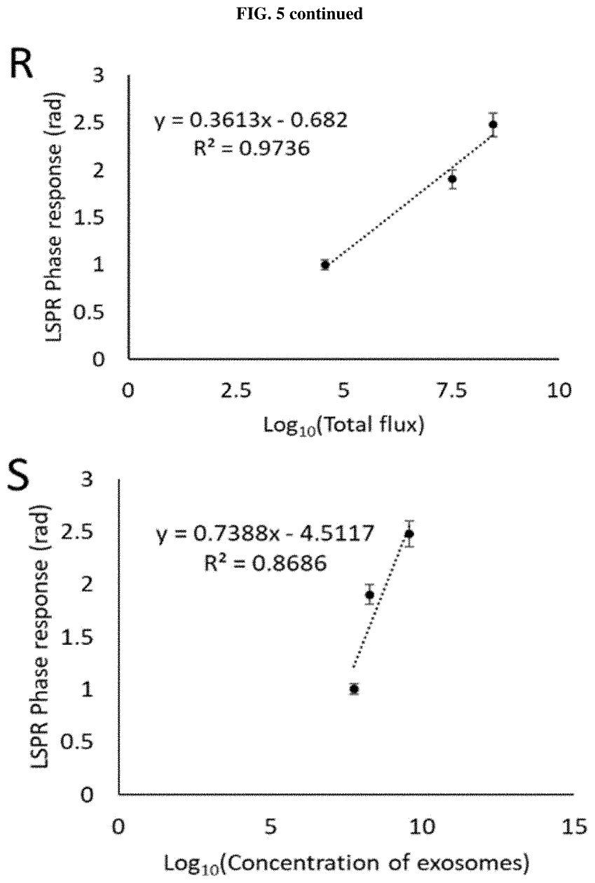

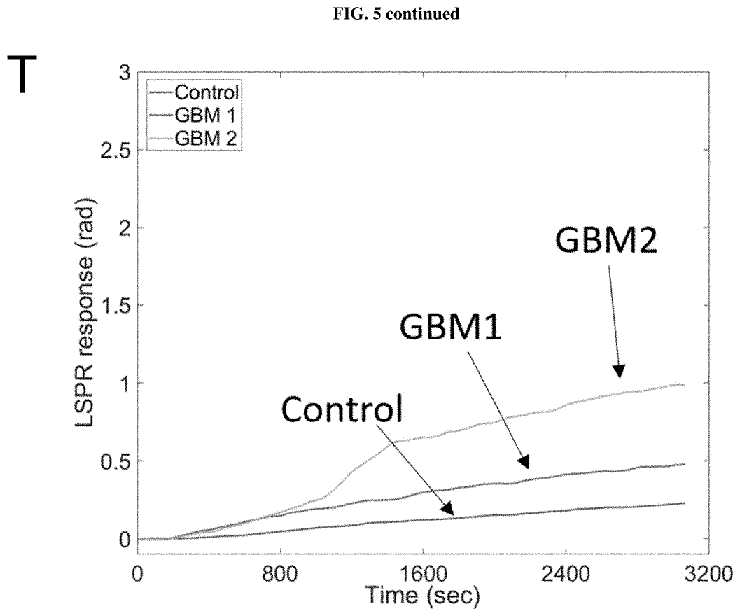

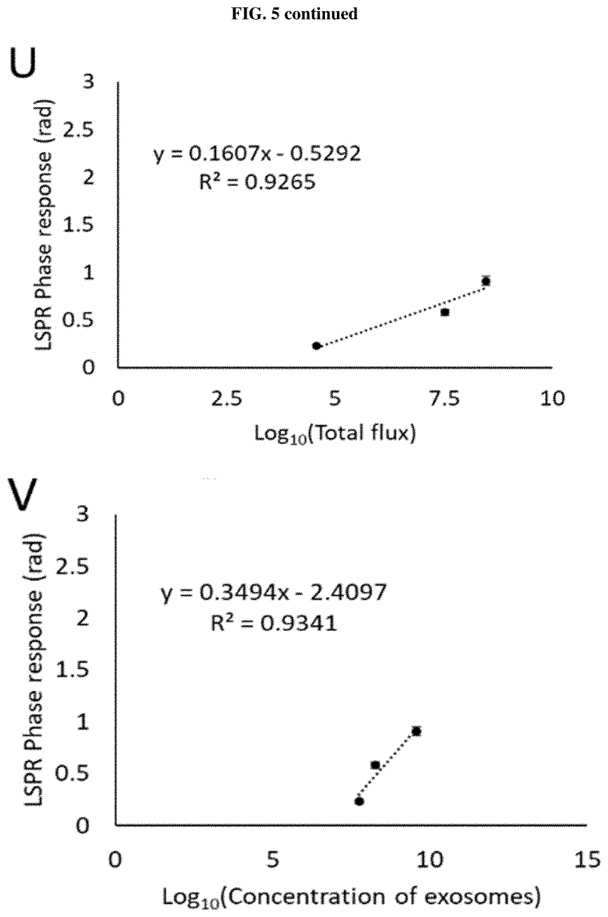

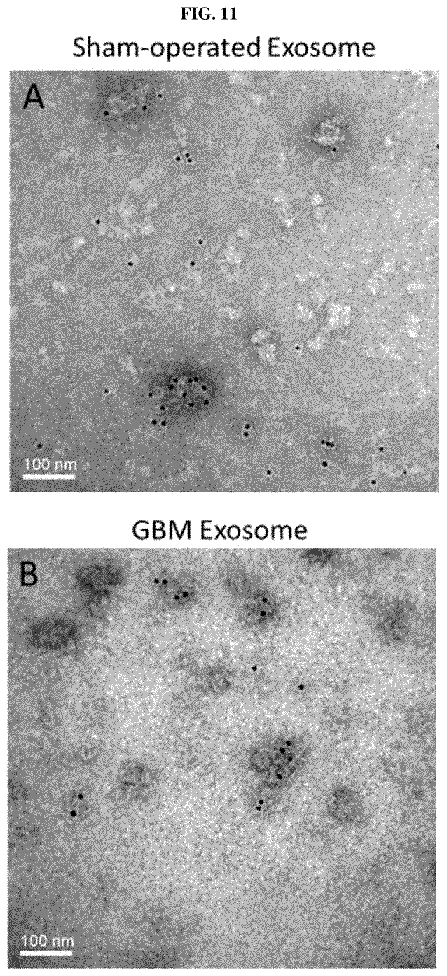

Firstly, GBM mouse model was developed by intracranial injection of U87 GMs (Luciferase) in six to eight-week-old severe combined immunodeficiency (SCID) mice, followed by histological H&E staining, immunofluorescent staining for MCT4 in the brain tissue of sham-operated and GBM mouse (FIG. 5A-L). Further, the bioluminescence imaging (BLI) was conducted to examine the development of tumour in sham-operated mouse—and GBM mice (GBM1 and GBM2)—brain (FIG. 5M), In BLI, the enhanced total flux (photons/s) of luminescence in GBM1 and GBM2 demonstrated the development of tumour with different size (FIG. 5N). Interestingly, the augmented release of exosomes from blood serum of GBM1 and GBM2 was found to be correlated with the tumour size based on BLI and total flux in GBM mouse brain (FIG. 5M-O). Immunogold EM confirmed the presence of MCT4 in sham-operated mouse—and GBM mouse—blood serum-derived exosomes with different number (FIG. 11A, B), which were further quantitatively examined via BAF Ag@AuNIs LSPR biosensor. The dynamic range and LOD of the BAF Ag@AuNIs LSPR biosensor for MCT4 in blood serum-derived exosomes from GBM mouse are 4×10⁻⁴ to 50 μg/mL and 0.4 ng/mL exosome concentration, respectively, (FIG. 5P) with the linear regression equation, y=0.3047×x+1.066 (R² 0.9576). This suggested that BAF Ag@AuNIs LSPR biosensor could detect the differential quantity of MCT4 in blood serum-derived exosomes with high sensitivity, supporting its use in testing blood-derived exosomes for the diagnosis of GBM in clinics.

Further, the exosomal concentration was tested to determine the best resolution capacity of the BAF Ag@AuNIs LSPR biosensor to distinguish GBM mice from sham-operated mouse. The LSPR phase response toward MCT4 was determined in two different concentrations (5 and 0.5 μg/mL) of exosomes isolated from sham-operated mice, GBM1, and GBM2 (FIGS. 5 Q and T). From these experiments, 5 μg/mL of exosomal concentration was found for distinguishing between sham-operated mouse, GBM1, and GBM2 via the detection of MCT4 in blood serum-derived exosomes with the best resolution (FIG. 5Q). Overall, a positive correlation was observed between the levels of augmented tumour size based on total flux of luciferase bioluminescence and LSPR phase response for MCT4 in exosomes (for 5 μg/ml, coefficient of determination (R²)=0.9736, and for 0.5 μg/mL, R²=0.9265)-derived from blood serum of sham-operated mouse, GBM1, and GBM2 (FIGS. 5 R and U) Similarly, a positive correlation was observed between released exosome concentration and the LSPR phase response for MCT4 in exosomes (for 5 μg/mL, R²=0.8686, and for 0.5 μg/ml, R²=0.9341)-derived from blood serum of sham-operated mouse, GBM1, and GBM2 FIGS. 5 S and V). This firmly supports use of a label-free minimally invasive BAF Ag@AuNIs LSPR biosensor for the detection of MCT4 in blood serum-derived exosomes for monitoring GBM formation and metabolic adaptation.

### Time Course In Vivo Monitoring of GBM Via the Detection of MCT4 in Blood Serum-Derived Exosomes by BAF Ag@AuNIs LSPR Biosensor

Due to lack of early diagnosis and effective treatment for glioblastoma, there exists a demand for developing a new technique to detect glioblastoma at an early stage with molecular and metabolic signatures for more precise diagnosis and treatment. Therefore, the minimally invasive label-free Au—Ag LSPR biosensor has been development and tested for the early detection of glioblastoma formation and malignancy as liquid biopsy.

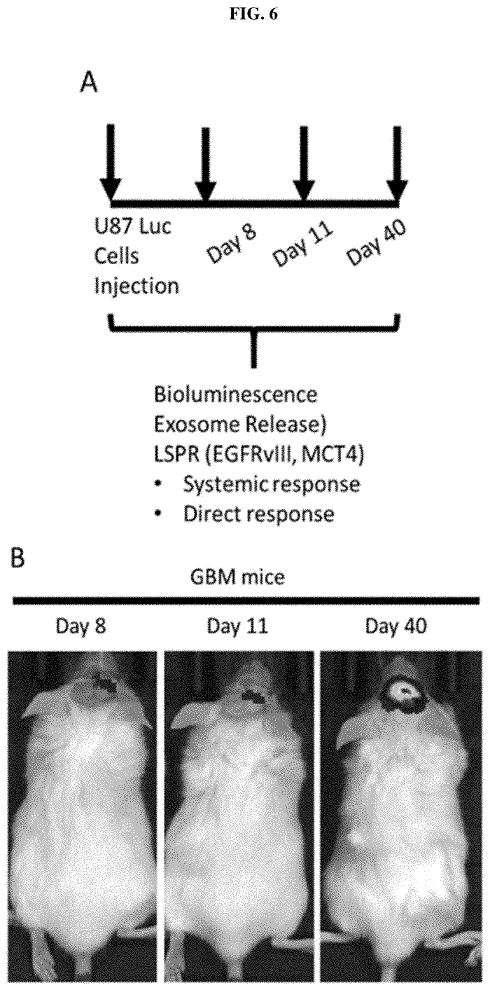

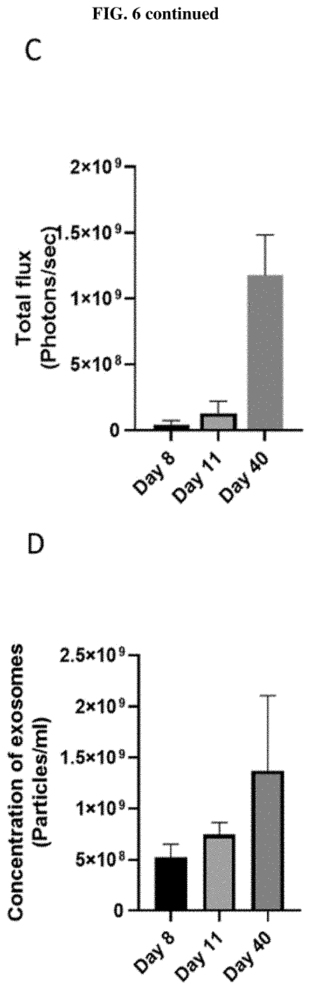

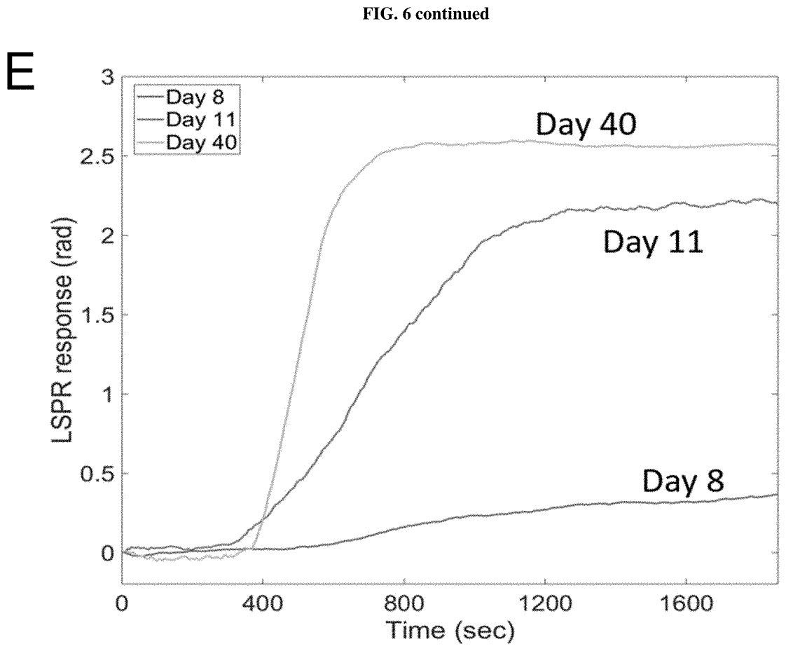

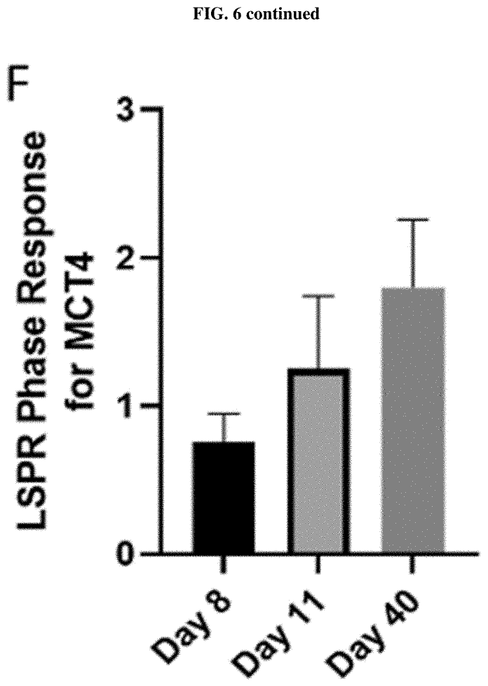

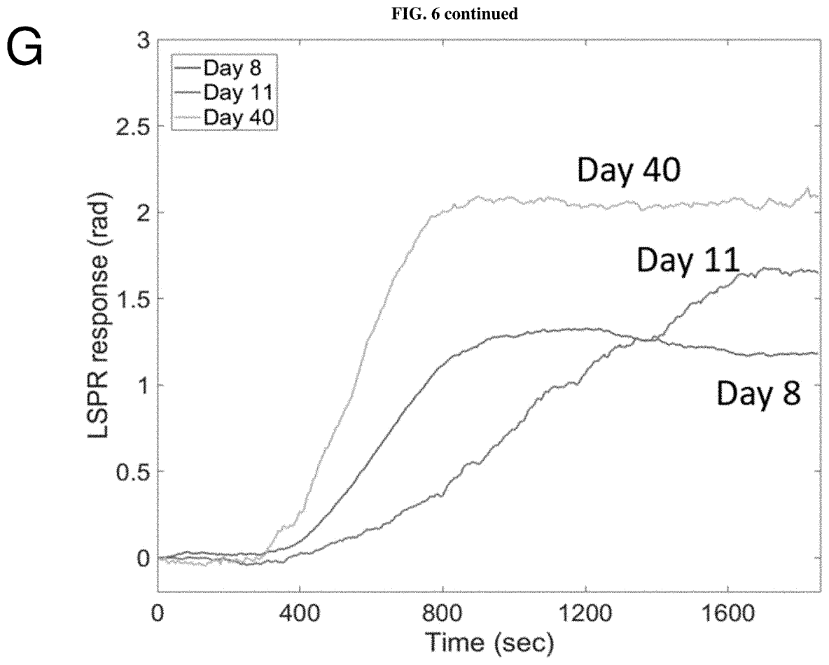

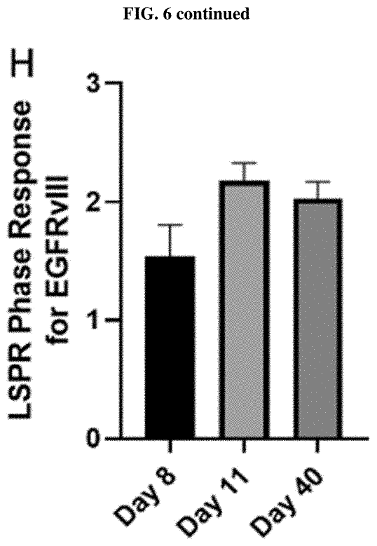

To examine the performance of the BAF Ag@AuNIs LSPR biosensor for early detection of GBM development and monitor the progress of tumour size in brain of GBM mouse, U87 luciferase GMs were injected intracranially in SCID mouse followed by tracking the development of GBM progression, exosomal release, and LSPR phase response for MCT4 towards blood serum-derived exosomes from GBM mouse and EGFRvIII-based immunocaptured blood serum-exosomes from GBM mouse (FIG. 6A). According to the BLI and measurement of total flux in the brain of GBM mouse at day 8, 11, and 40, the time-dependent progression of tumour was confirmed in GBM mouse (FIG. 6B, C). Along with the time-dependent tumour progression in GBM mouse, the increased release of exosomes from blood serum was also found (FIG. 6D). Further, time-dependent increase in MCT4 level was detected in exosomes from the blood of the GBM mice by the BAF Ag@AuNIs LSPR biosensor (FIG. 6E-H), indicating that the MCT4 level in blood serum-derived exosomes can be potentially used for monitoring tumour progression in GBM mouse. Nevertheless, the origins of exosomes from the blood serum are multiple because MCT4 is expressed in other cell types in the periphery. To determine whether GMs-derived exosomes in the blood serum contained high level of MCT4, they were isolated by a magnetic dynabeads-based immunocapture method using EGFRvIII ABs as described in Methods. Indeed, a number of GMs-derived exosomes were identified in the blood. High levels of MCT4 in immunocaptured GMs-derived exosomes were also detected and quantified by the BAF Ag@AuNIs LSPR biosensor (FIG. 6I-L). This further supports the utility of exosomal MCT4-based liquid biopsy for determining GBM malignancy through a LSPR biosensor.

The chip or biosensor, as disclosed herein, was developed to detect an infinitesimal amount of exosomal biomarkers. Self-assembly Ag nanoparticles located or dispersed on Au nano-islands (SAM Ag@AuNIs) sensor chip provide site-specific bio-conjunction of biotinylated antibodies on Ag nanoparticles located on the surface of Au nano-islands. When a sample solution containing an exceedingly low amount (infinitesimal amount) of exosomal biomarkers flows over the sensor chip, the biotinylated antibodies capture the exosomal biomarkers, effectively bringing them close to the sensor surface, and thus allowing the biomarker to be detected by the sensor. The sensing performance is also enhanced by plasmonic “hot spots” provided by the Au nano-islands structure, with the ability to detect. The SAM Ag@AuNIs LSPR biosensor sensitively detected CD63, exosome marker, and MCT4, a glioblastoma progression biomarker, in malignant GMs-derived exosomes in the dynamic range of 3.8×10⁴ to 50 μg/mL with limit of detection (LOD) of 0.38 ng/mL and 0.0014 to 500 μg/mL with LOD of 1.4 ng/mL, respectively. Furthermore, it detected the enhanced level of MCT4 in malignant hypoxic GMs-derived exosomes as well as increased MCT4 in the blood serum-derived exosomes of GBM mice in the dynamic range of 4×10⁴ to 50 μg/mL with LOD of 0.4 ng/ml. Finally, it was able quantify MCT4 in the isolated glioblastoma-derived exosomes from the blood of GBM mice by EGFRvIII-based immunocapture, indicating its suitability in minimally invasive monitoring of glioblastoma progression using, for example, liquid biopsies. With high sensitivity and selectivity in the label-free sensing for exosomal biomarkers, the biotinylated antibody functionalized (BAF) Ag@AuNIs LSPR biosensor provides early detection of GBM formation and progression.

Thus, in one example of an exosomal biomarker is MCT4. MCT4 is a protein that can be found in exosomes isolated from glioblastoma cells, and blood serum of glioblastoma (GBM)-injected mice. The disclosed biosensor detects the presence of MCT4 by taking advantage of the site-specific bio-conjunction on Ag nanoparticles taking advantage of the plasmonic “hot spots” at the edge between nano-islands and dielectric gaps.

In another example, the described biosensor was shown to detect CD63, a cell surface marker in glioblastoma-derived exosomes in the dynamic range of 3.3×10⁴ to 50 μg/ml with a limit of detection (LOD) of 0.38* ng/ml. The same biosensor was shown to detect MCT4 in glioblastoma-derived exosomes in the dynamic range of 0.0014* to 500 μg/mL with a limit of detection (LOD) of 1.4* ng/ml. The biosensor was shown to detect MCT4 in blood-derived exosomes obtained from glioblastoma injected mice in the dynamic range of 4×10⁴ to 50 μg/ml with a limit of detection (LOD) of 0.04 ng/ml. It was also shown that the biosensor disclosed herein was able to detect MCT4 in glioblastoma-specific exosomes from blood via EGFRvIII-based immunocapture, indicating utility of the biosensor for use with, for example, non-invasive liquid biopsy samples obtained from glioblastoma subjects. The sensitivities of the three biosensors is represented by the slopes of the calibration curves in FIG. 3B. The BAF Ag@AuNIs LSPR biosensor exhibits much higher slope than the other two, indicating that the BAF Ag@AuNIs LSPR biosensor has sensitivity of 1.71 and 2.12 times of those of the functionalized Au nano-islands (AuNIs) and functionalised Au—Ag bimetallic nano-islands (BMNIs), respectively. Thus, the exemplary biosensor disclosed herein showed fast and effective functionalisation, as well as increased sensitivity.

Thus, in one example, the biosensor disclosed herein detects an exosomal biomarker. Examples of an exosomal biomarker are, but are not limited to, exosomal surface markers, proteins involved in cancer progression. Examples of exosomal surface markers are, but are not limited to, CD9, CD63, and CD81. Examples of proteins involved in cancer progression are, but are not limited to, MCT4, MCT1, and CD147. Thus, in one example, the exosomal biomarker are, but are not limited to cell surface marker, surface presenting protein, receptor protein, and cancer specific mutant proteins. In another example, the exosomal biomarker are, but are not limited to, MCT1, MCT2, MCT4, CD147, CD44, EpCAM, CD34, CD44, CD20, CD166, CD133, CD24, CD45, and CD105

Sensitive Detection of MCT4 on GMs-Derived Exosomes by Biotinylated Antibody Functionalized (BAF Ag@AuNIs) LSPR Biosensor

It has been reported that higher expression of MCT4 is associated with the poor prognosis in glioblastoma patients. The same can be said, for example, cell lines such as, but not limited to the U87 glioblastoma cell line. Therefore, expression of MCT4 in glioblastoma-derived exosomes has been selected as a biomarker for diagnosis and prognosis of glioblastoma.

Thus, in one example, there is disclosed a method of detecting the presence or absence of exosomes, the method comprising detecting an exosomal biomarker in a sample obtained from a subject, the method comprising a biosensor, wherein the biosensor is functionalised to detecting the biomarker, wherein the binding of the biomarker to the biosensor results in a signal. Also disclosed herein is a system for detecting an exosomal biomarker as disclosed herein, the system comprising the biosensor as described herein and a detector. In another example, there is disclosed a method of detecting the presence or absence of an exosome in a sample, the method comprising contacting the sample suspected of comprising or comprising the exosome with a biosensor and detecting an exosomal biomarker on the exosome, wherein the biosensor is conjugated to a targeting agent selectively binding to and detecting the exosomal biomarker; wherein upon binding of the exosomal biomarker to the biosensor a signal is generated

Exosomes isolated from U87 glioblastoma cells were found to be approximately 80 to 300 nm in diameter (FIG. 3A), and circular in shape (FIG. 3B). The expression of CD63 positive immunogold dots shows that the presence and the use of CD36 as an exosomal marker (FIG. 3C). The sensing performance of the biotinylated antibody functionalized Ag nanoparticles locally attached on Au nano-islands (BAF Ag@AuNIs) LSPR biosensor toward glioblastoma-derived exosomes was examined using a real-time differential phase sensing system over the dynamic range of 3.8*×10⁴ to 50 μg/ml. The limit of detection (LOD) of the BAF Ag@AuNIs biosensor, as disclosed herein, was found to be 0.38* ng/ml (FIG. 3D) for CD63 in U87 glioblastoma-derived exosomes. The BAF Ag@AuNIs biosensor was found to possess higher sensitivity compared to functionalized Au—Ag bimetallic- (BMNIs) and functionalized Au nano-islands (AuNIs)-localised surface plasmon resonance (LSPR) biosensors in detecting the presence of CD63 in U87 glioblastoma-derived exosomes (FIG. 3E). This indicates a higher sensitivity (and thereby performance) of the described BAF Ag@AuNIs biosensor over biosensors in the art in detect glioblastoma-derived exosomes.

Thus, in one example, the exosome is produced by cancer cells. In other words, the exosomes disclosed herein are obtained from originate from a cancer cell. Such cancer cells can be, but are not limited to, cells originating from a cancer such as, but not limited to, brain cancer, metastatic brain cancer, lung cancer, prostate cancer, ovarian cancer, and other solid tumours Similar strategy can be applied to other cancer types by utilizing exosomes. In one example, the cancer is a glioblastoma. In another example, the exosome is produced by glioblastoma cells.

Further, the biotinylated antibody functionalized Ag nanoparticles locally attached on Au nano-islands (BAF Ag@AuNIs) localised surface plasmon resonance (LSPR) biosensor disclosed herein was utilized to analyse the expression of MCT4 in U87 glioblastoma-derived exosomes, which was also confirmed via the presence of MCT4 positive immunogold dots (FIG. 3F, G). The dynamic range and limit of detection (LOD) of the biosensor disclosed herein for MCT4 in U87 glioblastoma-derived exosomes are 0.0014* to 500 μg/ml and 1.4* ng/ml, respectively (FIG. 3F). This shows the ability of the disclosed BAF Ag@AuNIs LSPR biosensor to detect MCT4 in glioblastoma-derived exosomes and indicates application of the described biosensor in the analysis of, for example, liquid biopsy samples.

Thus, the disclosed exemplary biosensors showed sensitivity and selectivity with regard to U87 glioblastoma cell-derived exosomes and demonstrated use as a diagnostic tool for liquid biopsies of specific glioma.

The biotinylated antibody functionalized Ag nanoparticles locally attached on Au nano-islands (BAF Ag@AuNIs) localised surface plasmon resonance (LSPR) biosensor disclosed herein was shown to be selectivity for exosomal MCT4, as shown by way of the shift in phase response at two different concentrations of exosomes, namely at 0.5 μg/ml and 50 μg/ml. The phase response was found to be 2.956 rad and 1.649 rad (in other words, 2.956 rad versus 0.7306 rad at the concentration of exosomes to be 50 μg/ml; 1.649 rad versus 0.3124 rad at the concentration of exosomes to be 0.5 μg/ml), respectively. As expected from the data of immunogold electron microscopy, the retinal pigment epithelium (RPE) cell-derived exosomes also produced a weak phase response (FIG. 3H). Without being bound by theory, it is thought that the weak phase response was caused by the non-specific cross-reaction between, for example, the anti-MCT4 antibody used for nano-island functionalisation (also referred to as biotinylated antibody functionalised Ag nanoparticles locally attached on Au nano-islands (BAF Ag@AuNIs) and exosomal antigens other than MCT4. It is of note that these nonspecific bindings were effectively removed by, for example, flushing of the biosensor, resulting in dissociation (FIG. 3I).

Detection of MCT4 in normoxic and hypoxic glioblastoma-derived exosomes by BAF Ag@AuNIs LSPR biosensor

Hypoxia is one of the major hallmarks of the tumour microenvironment in cancers. To further examine the capacity of biotinylated antibody functionalized Ag nanoparticles locally attached on Au nano-islands (BAF Ag@AuNIs) localised surface plasmon resonance (LSPR) biosensor, as disclosed herein, to detect the malignant state of glioblastomas via the detection of exosomal MCT4, the U87 glioblastoma cells were exposed to normoxic and hypoxic conditions, and the level of MCT4 in normoxic and hypoxic glioblastoma-derived exosomes was detected. Under hypoxia, the expression of MCT4 was found to be increased on the membrane of U87 glioblastoma-derived exosomes (FIG. 4A-H). In addition, the concentration of MCT4 protein was also shown to be increased in U87 glioblastoma-derived exosomes under hypoxia compared to normoxia (FIG. 4I). It was also shown that the release of exosomes was also increased (around 25 times higher) under hypoxia compared to normoxia (FIG. 4J).

Conventional methods for the detection of protein, such as western blot, and ELISA, possess limitations for the study of exosomal protein. Therefore, biotinylated antibody functionalized Ag nanoparticles locally attached on Au nano-islands (BAF Ag@AuNIs) localised surface plasmon resonance (LSPR) biosensor as disclosed herein was developed. This biosensor was shown to be capable of detecting MCT4 in small amounts of glioblastoma-derived exosomes with a limit of detection (LOD) of 1.4* ng/ml (FIG. 3F, G), and was also shown to be capable of detecting MCT4 in glioblastoma-derived exosomes selectively, as shown by a reduced localised surface plasmon resonance (LSPR) phase response for MCT4 towards RPE-derived exosomes (FIG. 3H, I). Of note, the LSPR phase response for MCT4 for hypoxic glioblastoma-derived exosomes was shown to be higher compared to the normoxic glioblastoma-derived exosomes (FIG. 4K, L). This suggests the application of the biosensor disclosed herein a diagnostic tool for the detection of exosomal MCT4 to track the development, as well as malignant progression, of glioblastomas.

Thus, in one example, functionalisation of the biosensor disclosed herein is performed using, for example, but not limited to, antibodies, conjugated antibodies, or biotinylated antibodies. In other words, in one example, the targeting agent is, but is not limited to, an antibody, a biotinylated antibody, an antibody fragment (such as Fab, Fab′, F(ab′)2, and Fv), a single chain (ScFv)) a peptide, an aptamer, or a small molecule that is capable of selectively binding to a target of interest, such as, but not limited to, a carbohydrate, polynucleotide, lipid, polypeptide, protein, small molecule, and a cellular receptor.

Thus, in one example, the biosensor comprises Ag nanoparticles localised on Au nano-islands utilising localised surface plasmon resonance as a detection signal. In one example, the signal is measured as a phase shift.

In one example, the nano-islands are mono- or bimetallic. In another example, the nano-islands comprise noble metals. Examples of such noble metals are, but are not limited to, ruthenium (Ru), rhodium (Rh), palladium (Pd), osmium (Os), iridium (Ir), platinum (Pt), gold (Au), silver (Ag), and combinations thereof. In one example, the nano-islands and nanoparticles are made of the same or different noble metals.

In one example, the nano-islands obtained from or obtainable by dewetting or sputtering.

In one example, the method as disclosed herein comprises Ag nanoparticles locally attached on Au nano-islands, and wherein the biosensor utilises localised surface plasmon resonance as the signal.

In another example, the method is as disclosed herein, wherein nano-island are gold nano-islands, wherein the nanoparticles are silver nanoparticles, wherein the targeting agent is a biotinylated antibody, wherein the exosomal biomarker is MCT4, and wherein the signal is a phase shift.

Also disclosed herein is a system for detecting an exosomal biomarker according to the method as disclosed herein, the system comprising a biosensor comprising nanoparticles localised on nano-islands, wherein the nanoparticles and the nano-islands are of the same or different noble metals, wherein the nanoparticles are conjugated to a targeting agent selectively binding to and detecting the exosomal biomarker, and a detector. In one example, the system comprises gold nano-islands, and/or wherein the silver nanoparticles.

The collective oscillations of conduction band electrons which are in resonance with electromagnetic (EM) near-field stimulated by visible light in noble metal nanoparticles, known as localised surface plasmon resonance (LSPR), is important in real-time label-free biosensing. It has been shown that the locally enhanced electromagnetic field is confined to the vicinity of the nanostructure and that it can be further augmented at the optimal gaps between the nanostructure. Hence, high detection sensitivity can be achieved when the association and dissociation of biomarker molecules occur locally at the optimal distance between the nanostructure by producing enhanced localised surface plasmon resonance. It has also been demonstrated that silver (Ag) forms both physical and chemical bonding with functional groups such as —COOH and —SH of proteins, including antibodies (Abs). Also, silver produces effective plasmonic resonance stimulated by light at the blue end of the visible spectrum. Among the plasmonic materials in biosensing, gold (Au) is used owing to its merits including resistance to oxidation and corrosion, feasibility of fabrication to nanostructure, as well as suitable plasmonic resonance via the stimulation of light at the middle of the visible range.

Thus, disclosed herein is a biosensor comprising metallic nanoparticles conjugated to a targeting agent, wherein the metallic nanoparticles are on the surface of metallic nano-islands, wherein the nanoparticles are conjugated to a targeting agent, and wherein the nanoparticles and the nano-islands are of the same or different noble metals. In one example, such a biosensor comprises gold nano-islands and silver nanoparticles.

Optimization of Exosomal Concentration to Detect Resolution of Ag@AuNIs Localised Surface Plasmon Resonance (LSPR) Biosensor to Distinguish Glioblastoma-Injected Mice from Sham-Operated Mice by Detecting MCT4 in Blood Serum Exosomes

Firstly, a glioblastoma mouse model was developed by intracranially injecting U87 luciferase-expressing glioblastoma cells in six to eight-week-old severe combined immunodeficiency (SCID) mice. This was followed by bioluminescence assay to examine the development of tumour in sham-operated mice and the brains of glioblastoma-injected mice (GBM1 and GBM2; FIG. 5A). Together with bioluminescence imaging, the enhanced total flux in GBM1 and GBM2 showed the development of tumours with different sizes (FIG. 5B). It was noted that the increased release of exosomes in blood serum obtained from GBM1 and GBM2 was found to be correlated with the tumour size based on luminescence and total flux in glioblastoma-injected mice brains (FIG. 5A-C). The dynamic range and limit of detection (LOD) of the biotinylated antibody functionalized Ag nanoparticles locally attached on Au nano-islands (BAF Ag@AuNIs) localised surface plasmon resonance (LSPR) biosensor for MCT4 in blood serum-derived exosomes from glioblastoma-injected mice was 4×10⁻⁴ to 50 μg/ml and 0.04 ng/ml, respectively (FIG. 5D), indicating that the biosensor disclosed herein can detect MCT4 in blood serum-derived exosomes with high sensitivity. The described biosensor can there be used to test clinical samples in order to aid the diagnosis of glioblastoma.

Further, the exosomal concentration required to enable the highest resolution capacity of biotinylated antibody functionalized Ag nanoparticles locally attached on Au nano-islands (BAF Ag@AuNIs) localised surface plasmon resonance (LSPR) biosensor to distinguish glioblastoma-injected mice from sham-operated mice was determined. To this end, the localised surface plasmon resonance (LSPR) phase response for MCT4 at two different concentrations (5 and 0.5 μg/ml) of exosomes isolated from sham-operated mice, GBM1, and GBM2 (FIGS. 5 E and H), was determined. It was shown that exosomal concentrations of 5 μg/ml and 0.5 μg/ml showed good resolution capacity. In one example, an exosomal concentration of 5 μg/ml of was used for distinguishing between sham-operated mouse, GBM1, and GBM2, thereby enabling the detection of MCT4 in blood serum-derived exosomes (FIG. 5E). Overall, a positive correlation was observed between the levels of increased tumour size, based on total flux of luciferase bioluminescence and localised surface plasmon resonance (LSPR) phase response (for 5 μg/ml, coefficient of determination R²=0.9736, and for 0.5 μg/ml, R²=0.9265) for MCT4 in exosomes derived from blood serum of sham-operated mice, GBM1, and GBM2 (FIGS. 5 F and I). Similarly, a positive correlation was observed between released exosome concentration and the localised surface plasmon resonance (LSPR) phase response (for 5 μg/ml, R²=0.8686, and for 0.5 μg/ml, R²=0.9341) for MCT4 in exosomes derived from blood serum of sham-operated mice, GBM1, and GBM2 (FIGS. 5 G and J). This firmly supports the use of non-invasive BAF Ag@AuNIs LSPR biosensors for in the detection of MCT4 in blood serum-derived exosomes. This also has application for monitoring glioblastoma formation and metabolic adaptation.

Thus, the data provided herein shows that the disclosed biosensors are capable of detect mouse serum-derived exosomes with low limits of detection (LOD), thereby underlining identifying the capability of the described biosensors to quantify exosomes and exosomal proteins in biological fluids.

### Time-Dependent In Vivo Monitoring of Tumour Size Via the Detection of MCT4 in Blood Serum Exosomes by BAF Ag@AuNIs LSPR Biosensor

In order to examine the ability of the biotinylated antibody functionalized Ag nano-particles locally attached on Au nano-islands (BAF Ag@AuNIs) localised surface plasmon resonance (LSPR) biosensor disclosed herein in early detection of glioblastoma development (from, for example, liquid biopsy samples) and for monitoring the progress of tumour size in brain of glioblastoma-injected mice, luciferase-expressing U87 glioblastoma cells were injected intracranially in severe combined immunodeficiency (SCID) mouse. This was followed by tracking the development of glioblastoma progression, exosomal release, and LSPR phase response for MCT4 in blood serum-derived exosomes from glioblastoma mice and EGFRvIII-based immunocaptured blood serum-derived exosomes from glioblastoma mice (FIG. 6A). According to the bioluminescence imaging and measurement of total flux in brain of glioblastoma mice at day 8, 11, and 40 post-injection, the time-dependent progression of tumour was confirmed in glioblastoma mice (FIG. 6B, C). Along with the time-dependent tumour progression in glioblastoma mice, the release of exosomes from blood serum was also found (FIG. 6D). Further, a time-dependent increase in MCT4 level was detected in exosomes from the blood of the glioblastoma mice using the BAF Ag@AuNIs LSPR biosensor disclosed herein (FIG. 6E-H), indicating that MCT4 levels in blood serum-derived exosomes can be used for monitoring tumour progression in glioblastoma patients. It is noted that the origin of exosomes from the blood serum can be from multiple sources, as MCT4 is also expressed in other cell types in the periphery. To determine whether glioblastoma-derived exosomes in the blood serum contained high level of MCT4, glioblastoma-derived exosomes from blood serum were isolated using a magnetic dynabeads-based immunocapture method in conjunction with EGFRvIII antibodies. Indeed, a large number of glioblastoma-derived exosomes were identified in the blood. It is of note that high levels of MCT4 of immunocaptured glioblastoma-derived exosomes were also detected and quantified by the biosensor disclosed herein (FIG. 6I-L), further supporting use of exosomal MCT4-based liquid biopsy analysis for determining glioblastoma malignancy through the localised surface plasmon resonance (LSPR)-based biosensor disclosed herein.

Thus, the biosensor disclosed herein is a combination of stability, high sensitivity, and selectivity in label-free detection of target biomarkers. This, in conjunction with biotinylated antibody functionalised Ag nanoparticles locally attached on Au nano-islands (BAF Ag@AuNIs), for example, functionalised with biotinylated antibody (BAF), results in a biosensor that has application in early detection of glioblastoma formation and metabolic adaptation.

The method disclosed herein can be also used to determine if a subject is suffering from cancer, or if the subject is at risk of developing cancer. In one example, the cancer is glioblastoma. Once identified of having, for example, glioblastoma multiforme, or being at risk of developing glioblastoma, thought the detection of one or more exosomal biomarkers disclosed herein, a subject is treated with an anti-cancer compound or an anti-cancer therapy. In one example, the choice of anti-cancer compound or anti-cancer therapy is based on the type of cancer to be treated. For the example of glioblastoma, such anti-cancer compounds or anti-cancer therapies can be, but are not limited to, alone, or in combination, resection, surgery, radiation, as well as chemotherapeutics (such as, but not limited to, temozolomide, lomustine, bevacizumab, and combinations thereof).

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

As used in this application, the singular form “a,” “an,” and “the” include plural references unless the context clearly dictates otherwise. For example, the term “a genetic marker” includes a plurality of genetic markers, including mixtures and combinations thereof.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

### Chemicals

11-mercaptoundecanoic acid (11-MUA), N-hydroxysuccinimide (NHS), N-(3-dimethylaminopropyl)-N′-ethylcarbodiimide (EDC) and ethanolamine (EA) and biotin were purchased from Sigma-Aldrich (Saint Louis, USA). 0.01M phosphate buffered saline (PBS, Sigma-Aldrich, pH 7.4 at 25° C., containing 0.138M NaCl; 0.0027M KCl) was employed as the running buffer in the experiments. pH 7.4 was maintained for the whole processes of functionalization and detection.

### Synthesis of the Self-Assembly Ag Nanoparticles Locally Attached on Au Nano-Islands (SAM Ag@AuNIs) Localized Surface Plasmon Resonance (LSPR) Sensor Chip

A two-step self-assembly thermal annealing method was employed to fabricate the SAM Ag@AuNIs chip on dielectric BK7 substrate. In detail, cleaned BK7 glass slides (35 mm×25 mm×2 mm) were initially sputtered with Au film, and then annealed at 550° C. for 3 hours in air. This resulted in a self-assembly Au nano-islands structure on the surface of the BK7 substrate. This surface was then sputtered with a film of Ag, followed by a second thermal annealing process at 150° C. for 30 minutes in air. The end result was Ag nanoparticles attached on the surface of the Au nano-islands. The thickness of sputtered Au and Ag film was 5.5 nm and 1.8 nm, controlled by sputtering time. The elevation rate of the annealing temperature was fixed at 15° C./minute.

Functionalization of SAM Ag@AuNIs with Biotinylated Antibody

Biotinylated antibody was utilized to functionalise the self-assembly silver (Ag) nanoparticles locally attached on gold (Au) nano-islands (also referred to herein as “SAM Ag@AuNIs”) to obtain biotinylated antibody functionalised Ag nanoparticles locally attached on Au nano-islands (BAF Ag@AuNIs) biosensor for plasmonic biosensing. In detail, phosphate-buffered saline (PBS) was injected into a probe cell of the biosensor to stabilise the surface. Subsequently, the biotinylated antibody was injected for 10 minutes, after which the substrate was flushed with PBS to remove any unbound biotinylated antibody. 1 μg/ml biotin solution and 0.1M ethanolamine were injected for 10 minutes and 3 minutes, respectively, to block the unbound surface. Biotin solution was prepared by directly dissolving biotin into PBS to obtain a biotin solution at the desired concentration.

Detection of Exosomal CD63 and MCT4 with Biotinylated Antibody Functionalised Ag Nanoparticles Locally Attached on Au Nano-Islands (BAF Ag@AuNIs) Plasmonic Biosensor

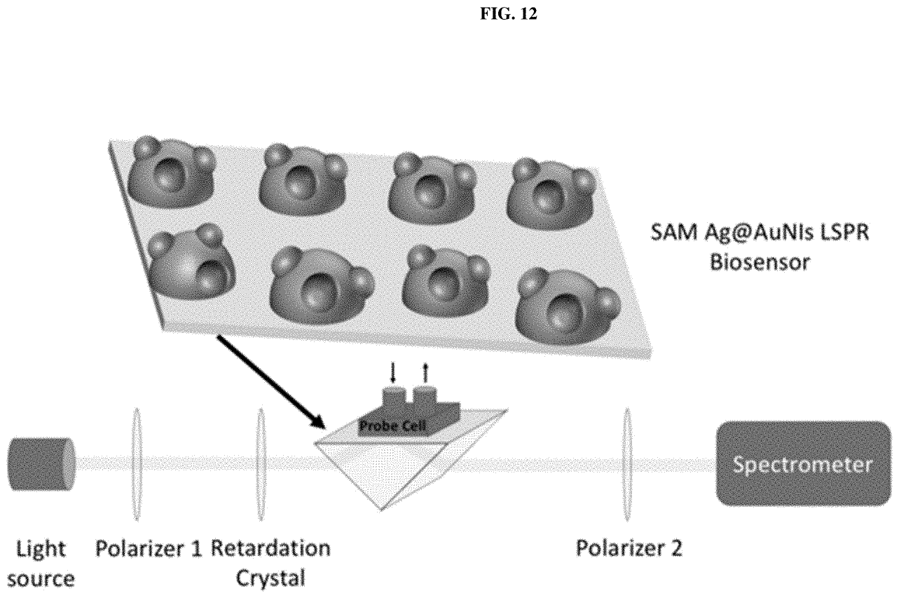

To perform the label-free detection of exosomes with the biotinylated antibody functionalised Ag nanoparticles locally attached on Au nano-islands (BAF Ag@AuNIs) biosensor, a common-path white light interferometric system was used. Phosphate-buffered saline (PBS) was initially injected into the probe cell of the biosensor for 10 mins to build the detection baseline. Then, the glioblastoma-derived exosomes or the mouse serum-derived exosomes were injected into the probe cell of the biosensor (FIG. 12). Subsequently, phosphate-buffered saline (PBS) was injected to flush the non-specific bonded items from the biosensing interface. All the injection was implemented using a peristaltic pump at a constant rate of 30 μl/min to avoid damaging the nanostructure by flushing. The flow-in and flow-out ends on probe cell are vertical aligned to generate turbulent mixing to reduce the mass transfer resistance. Windowed Fourier transform (WFT) method was utilized to extract the differential phase response in real-time scheme.

### Characterisation of Self-Assembly Ag Nanoparticles Locally Attached on Au Nano-Islands (SAM Ag@AuNIs) and Biotinylated Antibody Functionalised Ag Nanoparticles Locally Attached on Au Nano-Islands (BAF Ag@AuNIs) and Captured Glioblastoma-Derived Exosomes

The surface morphology of as-synthesized Ag nanoparticles locally attached on Au nano-islands and nano-island sizes were characterized by atomic force microscopy (AFM, Veeco di Multimode V) and scanning electron microscopy (SEM, thermo scientific Quattro S). Phase mode of atomic force microscopy was employed to distinguish between the gold (Au) nano-islands and the silver (Ag) nanoparticles. Ag@AuNIs structure, biotinylated antibody functionalised Ag nanoparticles locally attached on Au nano-islands (BAF Ag@AuNIs) interface and exosome-adsorbed on biotinylated antibody functionalised Ag nanoparticles locally attached on Au nano-islands (BAF Ag@AuNIs) structure were scanned using an atomic force microscopy tapping mode. The functionalized biotinylated antibody functionalised Ag nanoparticles locally attached on Au nano-islands (BAF Ag@AuNIs) biosensor chips were flushed with deionised (DI) water and dried in a vacuum before conducting the atomic force microscopy scanning Similarly, the biotinylated antibody functionalised Ag nanoparticles locally attached on Au nano-islands (BAF Ag@AuNIs) chips with adsorbed exosomes were also dried in a vacuum just before the atomic force microscopy scanning In phase-mode AFM, the phase is correlated to the oscillation frequency of the AFM tip, when it taps along the surface with locations of different hardness. Therefore, it can assist to distinguish surface with different hardness.

Selective Functionalisation of the Ag Nanoparticles of SAM Ag@AuNIs LSPR Sensor Chip with Biotinylated Antibodies

Biotinylated antibodies were utilized to functionalise the SAM Ag@AuNIs chips for plasmonic biosensing. In detail, PBS buffer was injected initially into the probe cell to stabilize the surface of the chip. Subsequently, biotinylated Abs were injected in the probe cell for 10 minutes to functionalize the surface of the sensor chip. Then, the probe cell was flushed by PBS buffer to remove unbonded biotinylated Abs in the surface of the chip. At this stage the biotinylated antibody functionalized Ag nanoparticles locally attached on Au nano-islands (BAF Ag@AuNIs) LSPR biosensor was made, ready for sensing. 1 μg/mL biotin solution and 0.1M ethanolamine were injected for 7 minutes and 3 minutes, respectively, to block the unbonded surface of the chip. The biotin solution was prepared by directly dissolving the appropriate amount of biotin into PBS solution. The concentration of Abs used was generally 0.5 mg/mL to enhance the surface receptors (Abs) density of the SAM Ag@AuNIs chip, resulting in increasing receptor-ligand interaction, thus enabling sensitive detection of tiny amount of exosomal surface antigens. All injections were implemented by a peristaltic pump at a constant rate of 30 μl/min to avoid nanostructure damage.

### Cell Culture

U87 glioblastoma cells were cultured and maintained in Dulbecco's modified essential medium with high glucose (DMEM-H) (Gibco, Cat #10569-010) supplemented with 10% fetal bovine serum (FBS) (Gibco, Cat #10270-106) and 1% penicillin-streptomycin (Pen-Strep) (Gibco, Cat #15140-122) at 37° C. with 5% CO₂ in a humidified incubator. U87 luciferase glioblastoma cells (from Dr. Jun Sun Yoo's Lab, HTI, The Hong Kong Polytechnic University) were cultured in minimal essential medium (MEM) with Earle's balanced salt solution (EBSS) (GE Life Sciences, Cat #SH30024.01) supplemented with 10% FBS and 1% pen-strep at 37° C. under 5% CO2.

### Induction of Hypoxia to U87 GMs

To induce hypoxia, U87 glioblastoma cells were incubated in the chamber (Smartor 118 hypoxia chamber) supplied with a gas mixture, comprising 1% O₂, 5% CO₂, and 94% N₂ at 2 pounds per square inch, at 37° C. for 24 hours.

### Animal

All animal experiments followed the Institutional Animal Care and Use Committee (IACUA) guidelines and were approved by the Animal Research Ethics Sub-Committee in the City University of Hong Kong and Department of Health, Government of the Hong Kong Special Administrative Region. Mouse experiments were performed using six to eight-week-old SCID mice (The Jackson Laboratory, US). Free access to water and food ad libitum were provided for mice and they were housed under a 12-hr-light/12-hr-dark cycle in the Laboratory Animal Research Unit (LARU) of the City University of Hong Kong.

### Orthotropic Xenograft Mouse Model of GBM

Six to eight-week-old SCID mice were anesthetized by ketamine/xylazine (up to 100 mg/kg and 10 mg/kg, Alfamedic, Cat #013004 and 013006 respectively). After ensuring anesthesia by checking body reflexes, they were fixed on a stereotactic frame. The injection coordinates for the orthotropic model and resection model were 0.1 mm posterior, 2.3 mm lateral from the Bregma and 2.3-2.8 mm deep from the outer border of the cranium, respectively. Briefly, mouse skulls were first broken with a micro drill, and a cavity was created on the cranium. Then, 2 μl of 2×10⁴ U87-luciferase GMs were injected in the right frontal lobe using a 26-G needle in a 10 μl Hamilton syringe with a flow rate of 0.2 μl per min.

### In Vivo Bioluminescence Assay

Both glioblastoma- (GBM-) and wild type-mice (three mice per group) were first anesthetized with 2% isoflurane/O₂ in the induction chamber. 15 μg/mL sterile D-luciferin, potassium salt (Gold Biotechnology, Cat #LUCK-250) in Dulbecco's phosphate-buffered saline (DPBS) (Gibco) was then injected subcutaneously at a volume of 10 μl/g of body weight. Sedated mice were then placed in the 37° C.-imaging chamber of the IVIS Spectrum In Vivo Imaging System (PerkinElmer) following a 10-minute incubation. Images were acquired with the built-in camera of the IVIS System. Region of interest (ROI) for bioluminescence was measured in terms of total flux at a unit of photons per second.

Isolation of Exosomes from Glioblastomas and Blood Serum

For the isolation of glioblastoma- (GM-) derived exosomes, FBS-free conditioned medium from cultured U87 glioblastoma cells, which is priorly incubated 1-2 days before collection, was harvested, and exosomes then were isolated by using the Total Exosome Isolation (TEI) kit for cell culture (Invitrogen) as per the manufacturer's protocol. For the isolation of exosomes from blood serum, blood was first taken via the tail vein of both GBM- and wild type-mice. Exosomes were isolated by using the TEI kit for blood serum (Invitrogen) as per the manufacturer's protocol. All isolated exosomes were quantified based on the protein amount as determined by Pierce BCA Protein Assay Kit (Thermo Fisher Scientific).

### Analysis of Size Distribution and Concentration of Exosomes

Size distribution and concentration of isolated exosomes were determined by Nano-tracking Analysis (NTA) using Malvern NanoSight NS300 instrument as described previously.

Isolation of Glioblastoma-Derived Exosomes from Blood Serum Using a EGFRvIII-Based Immunocapture Method

Glioblastoma-derived exosomes were isolated from the blood serum of GBM mice by using a EGFRvIII-based immunocapture method as described previously.35 Briefly, for EGFRvIII AB (Bioss, Cat #bs-2558R) based-immunocapture, at a ratio of 1 μg of Abs per 100 μl of beads were coupled to Pierce Protein A Magnetic Beads (Dyna beads), followed by incubation at 4° C. overnight. Beads were then washed three times with 500 μl of PBS containing 0.001% Tween (PBS-Tween), resuspended in 500 μl of the same buffer, to which exosomes (2×1010) were added, followed by overnight incubation at 4° C. with rotation. Bead-bound exosomes were collected and washed three times in 500 μl of PBS-Tween. Finally, exosomes were eluted with high-salt buffer and washed again and centrifuged at 100,000 g for 1 hour at 4° C.

### Transmission Electron Microscopy

Size and morphology of glioblastoma-derived exosomes were investigated by TEM analysis as described previously. Briefly, exosome pellet was suspended in 4% paraformaldehyde (PFA) and deposited onto formvar carbon-coated EM grids (Beijing Zhongjingkeyi Technology Co., Ltd, Cat #BZ1102XX). 2% uranyl acetate (UA) was used as a negative stain and processed grids were viewed under a Transmission Electron Microscope (TEM) (FEI/Philips Tecnai 12 BioTWIN).

### Immuno Gold Electron Microscopy

The immunogold labelling of exosomes with antibodies was carried out as described previously. In brief, exosome-coated grids with 5% BSA were transferred to a drop of antibodies (1:50 dilution of anti-CD63 antibodies, anti-EGFRvIII antibodies, anti-MCT4 antibodies) in PBS/0.5% BSA and incubated for 24 hours at 4° C. 10 nm gold-labelled nanoparticles (Abcam, Cat #ab27234) in PBS/0.5% BSA was used as the secondary antibodies and incubated for 1 hour at room temperature. 2% UA was used as a negative stain substrate and the stained image of exosomes were viewed under TEM.

Detection of Exosomal Surface Biomarkers with the Biotinylated Antibody Functionalized (BAF) Ag@AuNIs LSPR Biosensor

To perform the label-free detection of exosomal surface antigens with the biotinylated antibody functionalized (BAF) Ag@AuNIs LSPR biosensor, the common-path white light interferometric system was used. PBS buffer was initially injected into the sensing probe cell to build the detection baseline for 1 hour. Then, glioblastoma-derived exosomes, or mouse blood serum-derived exosomes were injected into the probe cell for 13 minutes. The concentration of exosomes was used as mentioned in the respective result section. Subsequently, PBS buffer was injected into the probe cell to flush and get rid of non-specific bonded items on the biosensing interface of the flow cell. All the injections were implemented by a peristaltic pump at a constant rate of 30 μl/min to avoid nanostructure damage by strong flow and flushing of sample and buffer. The flow-in and flow-out ends of the probe cell were vertically aligned to generate turbulent mixing to reduce the mass transfer resistance. Windowed Fourier transform (WFT) method was utilized to extract the differential phase response in real-time scheme.

### Detection of Biotinylated Abs Functionalized, and Exosomes Captured on SAM Ag@AuNIs LSPR Sensor Chip by Atomic Force Microscopy

The topography of SAM Ag@AuNIs LSPR sensor chip, and its functionalized state with biotinylated antibodies, and its state with GMs-derived exosomes captured via BAF Ag@AuNIs sensor chip were evaluated by atomic force microscopy (AFM, Veeco di Multimode V). The BAF Ag@AuNIs LSPR sensor chip was flushed with deionized water and dried in vacuum before conducting the AFM scanning Similarly, the BAF Ag@AuNIs LSPR sensor chip with captured exosomes was also flushed with deionized water and dried in vacuum before the AFM scanning.

### Statistical Analysis

The results in FIGS. 3D, E, G, 4J, L, 5B, C, F, G, I, J, and 6 C, D, F, H were presented as average±standard error (S.E.) of three replicates. Student's t-test was used for the comparison of experimental group(s) with the control group. *P-value <0.05, **P-value <0.001 were considered as significant.

1. A method of detecting the presence or absence of an exosome in a sample, the method comprising contacting the sample suspected of comprising or comprising the exosome with a biosensor and detecting an exosomal biomarker on the exosome, wherein the biosensor is conjugated to a targeting agent selectively binding to and detecting the exosomal biomarker; wherein upon binding of the exosomal biomarker to the biosensor a signal is generated.

2. The method of claim 1, wherein the exosomal biomarker is selected from the group consisting of cell surface marker, surface presenting protein, receptor protein, and cancer specific mutant proteins.

3. The method of claim 1, wherein the exosomal biomarker is selected from the group consisting of MCT1, MCT2, MCT4, CD147, CD44, EpCAM, CD34, CD44, CD20, CD166, CD133, CD24, CD45, and CD105.

4. The method of claim 1, wherein the exosome is produced by a cancer cell.

5. The method of claim 4, wherein the cancer cell is a cell originating from a cancer selected from the group consisting of brain cancer, metastatic brain cancer, lung cancer, prostate cancer, ovarian cancer, and other solid tumours.

6. The method of claim 5, wherein the cancer is a glioblastoma.

7. The method of claim 1, wherein the biosensor comprises nanoparticles localised on nano-islands, wherein the nano-islands and nanoparticles are made of the same or different noble metals.

8. The method of claim 7, wherein the noble metals are selected from the group comprising ruthenium, rhodium, palladium, osmium, iridium, platinum, gold, silver, and combinations thereof.

9. The method of claim 1, wherein the biosensor comprises Ag nanoparticles locally attached on Au nano-islands, and wherein the biosensor utilises localised surface plasmon resonance as the signal.

10. The method of claim 7, wherein the nano-islands are obtained by dewetting or sputtering.

11. The method of claim 1, wherein the targeting agent is selected from the group consisting of antibodies and biotinylated antibodies.

12. The method of claim 1, wherein the signal is measured as a phase shift.

13. The method of claim 1, wherein if the presence of exosomes is detected, the subject is treated with an anti-cancer compound or an anti-cancer therapy.

14. The method of claim 1, wherein the biosensor comprises nano-islands and nanoparticles, wherein the nano-island are gold nano-islands, wherein the nanoparticles are silver nanoparticles, wherein the targeting agent is a biotinylated antibody, wherein the exosomal biomarker is MCT4, and wherein the signal is a phase shift.

15. A system for detecting an exosomal biomarker according to the method as disclosed in claim 1, the system comprising a biosensor comprising nanoparticles localised on nano-islands, wherein the nanoparticles and the nano-islands are of the same or different noble metals, wherein the nanoparticles are conjugated to a targeting agent selectively binding to and detecting the exosomal biomarker, and a detector.

16. The system of claim 15, wherein the nano-islands are gold, and/or wherein the nanoparticles are silver nanoparticles.

17. A biosensor comprising metallic nanoparticles conjugated to a targeting agent, wherein the metallic nanoparticles are on the surface of metallic nano-islands, wherein the nanoparticles are conjugated to a targeting agent, and wherein the nanoparticles and the nano-islands are of the same or different noble metals.

18. The biosensor of claim 17, wherein the nano-islands are gold, and/or wherein the nanoparticles are silver nanoparticles.
